(19) United States
(12) Patent Application Publication
(10) Pub. No.: US 2023/0248809 A1
(43) Pub. Date: Aug. 10, 2023

(54) METHODS, DEVICES, AND SYSTEMS FOR TREATING LENS PROTEIN AGGREGATION DISEASES

(71) Applicant: Alan Neil Glazier, Rockville, MD (US)

(72) Inventor: Alan Neil Glazier, Rockville, MD (US)

(21) Appl. No.: 17/666,424

(22) Filed: Feb. 7, 2022

(51) Int. Cl. A61K 38/46; A61K 31/7105; A61K 31/711; A61K 38/45; A61K 38/17; A61K 38/48; A61K 31/10; A61K 38/06; A61K 45/06; A61K 48/00; A61P 27/06

(52) U.S. Cl. CPC A61K 38/465; A61K 31/7105; A61K 31/711; A61K 38/45; A61K 38/177; A61K 38/48; A61K 31/10; A61K 38/06; A61K 45/06; A61K 48/00; A61P 27/06

(57) ABSTRACT

Disclosed herein are methods, devices, and systems for treating lens protein aggregation diseases by reducing the formation of proteins responsible for crowding, compacting, and/or causing increased internal lens pressure. The methods, devices, and systems for treating lens protein aggregation disease may further maintain the circulating flux of ions into and out of the lens. Specifically disclosed herein are embodiments that genetically modify cellular machinery that produces one or more of these proteins. This may be achieved by, for example, one or more gene editing methods that delete (or “knock out”) one or more genes, or combinations thereof, responsible for the production of proteins responsible for crowding, compacting, and/or causing increased internal lens pressure.

### FIELD

The application relates generally to methods, devices, and systems for treating lens protein aggregation diseases. In particular, the application relates to novel methods, devices, and systems for reducing the formation of proteins responsible for crowding, compacting, and/or causing increased internal lens pressure.

### REFERENCE TO SEQUENCE LISTING

This application contains a Sequence Listing that has been submitted electronically in ASCII format and is hereby incorporated by reference in its entirety. The Sequence Listing was created on Mar. 14, 2022, has a file name of BGLAZ-P002-US_ST25.txt, and is 26 kilobytes in size.

### BACKGROUND

Mammalian lens protein aggregation diseases affect the human eye, including presbyopia and cataract. For the average healthy (e.g., non-diabetic, non-smoking) individual, presbyopia can manifest clinically in the early 40's as difficulty seeing objects at close range. However, the processes that lead to presbyopia often begins decades before any clinical symptoms are evident. One of the aforementioned processes leads to a dramatic increase in lens stiffness as an individual ages. For instance, the nucleus, which is a part of the eye lens, becomes approximately 500- to 1000-fold stiffer over the average person's lifetime. Generally, the onset of symptoms associated with presbyopia and other lens aggregation diseases are attributed to the loss of natural enzymatic and antioxidant protection in the eye against, for instance, ultraviolet A (UVA) and ultraviolet B (UV B) radiation, with a concurrent increase in the production of photochemically active chromophores (oxidants).

Accordingly, the key cause of presbyopia and, ultimately, cataractogenesis, is believed to be multifactorial, influenced by a combination of endogenous and exogenous oxidation. Endogenous oxidation occurs via internal mechanisms (e.g., intraocular photochemical generation of free radicals and other oxidants), while exogenous oxidation may be due to exposure to environmental causes (e.g., an increased exposure over an individual's lifespan to short wavelength and ultraviolet (UV) radiation, chemical ingestion (such as smoking), diabetes, and the like). However, the theory for oxidation as the root cause of presbyopia and other mammalian lens aggregation diseases cannot alone account for changes that result in protein aggregation (e.g., an increase in lens pressure, a decrease in lens flexibility). Such changes may play a more significant role in lens aggregation diseases than currently acknowledged.

Given the foregoing, there exists a significant need for novel technology that manages (e.g., maintains and/or reduces) internal lens pressure, and specifically intralenticular pressure, thus reducing onset and/or treating lens protein aggregation diseases, such as, for instance, presbyopia and cataracts.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In general, the present disclosure is directed towards methods, devices, and systems for reducing internal lens pressure and, in particular, intralenticular pressure (i.e., pressure inside the capsule of the lens). In particular, the disclosure relates to methods, devices, and systems for reducing the presence and/or formation of proteins responsible for crowding, compacting, and/or causing increased internal lens pressure (e.g., increased intralenticular pressure). The disclosure further relates to methods, devices, and systems that maintain circulating flux of ions into and out of the lens (e.g., reducing accumulation of calcium).

In at least one embodiment, a method for genetically modifying cellular machinery that produces one or more of the aforementioned proteins is disclosed. Specifically one or more gene editing methods may be used to delete (or “knock out”) one or more genes, or combinations thereof, responsible for the production of proteins responsible for crowding, compacting, and/or causing increased internal lens pressure.

In at least a further embodiment, a method is disclosed for reducing the production of lens crystallin proteins, including, but not limited to, crystallin proteins produced by the germinative region of the anterior epithelium of the mammalian lens. Specifically, one or more gene editing processes may be used, including, but not limited to, CRISPR (clustered regularly interspaced short palindromic repeats), in order to delete or downregulate various genes. These genes include, but are not limited to, the epithelial αB lens crystallin promoter gene, the epithelial βA4 lens crystallin promoter gene, the epithelial βα3/α3 lens crystallin promoter gene, the epithelial ββ2 lens crystallin promoter gene, the epithelial ββ2P1 lens crystallin promoter gene, the p38 mitogen-activated protein kinase (MAP kinase or MAPK) gene, and/or the Crystallin-50 (Cx50) gene.

In at least an additional embodiment, a method is disclosed that comprises administering gene therapy to a patient with one or more lens conditions (e.g., presbyopia, cataract, glaucoma, accumulation and/or aggregation of misfolded proteins in the lens, loss of clearance of misfolded proteins in the lens, oxidation of misfolded proteins in the lens, decrease in lysosomal activity in the lens, cell death in the lens) or symptoms thereof. The method may further comprise administering the aforementioned gene therapy to the patient before the one or more lens conditions occur. The gene therapy may comprise donor deoxyribonucleic acid (DNA) and/or a CRISPR/Cas9 complex to deliver the donor DNA and/or the CRISPR/Cas9 complex inside the cells of the patient.

One or more compounds may also be administered to the patient, such as, for instance, an inhibitor that reduces, inhibits, and/or eliminates expression of one or more genes herein. The inhibitor may include, for instance, a gene knockdown construct and/or artificial nucleic acid molecule (e.g., small interfering RNA (siRNA), microRNA (miRNA), short hairpin RNA (shRNA), antisense RNA, single guide RNA (sgRNA)), and/or a gene editing nuclease knockdown (e.g., via CRISPR/Cas9) of activating transcription factor 4 (ATF4) and/or C/EBP homologous protein (CHOP).

The one or more compounds may also contain synergistic amounts of one or more protein chaperones to assist in proper protein folding, one or more pharmaceutically acceptable excipients known in the art, and combinations thereof.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 is a diagram of a method for reducing formation of proteins responsible for crowding, compacting, and/or causing increased lens pressure, according to at least one embodiment of the present disclosure.

FIG. 2 is a diagram of a method for maintaining and/or reducing internal lens pressure by increasing gap junction coupling and/or reducing calcium accumulation, according to at least one embodiment of the present disclosure.

FIG. 3 is a diagram of a method for reducing lens hydrostatic pressure, according to at least one embodiment of the present disclosure.

FIG. 4 is a diagram of a method for reducing formation of cataracts, according to at least one embodiment of the present disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms first, second, etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of”. Similarly, the term “consisting essentially of” is intended to include embodiments encompassed by the term “consisting of”. Although having distinct meanings, the terms “comprising”, “having”, “containing” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

In general, the word “instructions,” as used herein, refers to logic embodied in hardware or firmware, or to a collection of software units, possibly having entry and exit points, written in a programming language, such as, but not limited to, Python, R, Rust, Go, SWIFT, Objective C, Java, JavaScript, Lua, C, C++, or C #. A software unit may be compiled and linked into an executable program, installed in a dynamic link library, or may be written in an interpreted programming language such as, but not limited to, Python, R, Ruby, JavaScript, or Perl. It will be appreciated that software units may be callable from other units or from themselves, and/or may be invoked in response to detected events or interrupts. Software units configured for execution on computing devices by their hardware processor(s) may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. Generally, the instructions described herein refer to logical modules that may be combined with other modules or divided into sub-modules despite their physical organization or storage. As used herein, the term “computer” is used in accordance with the full breadth of the term as understood by persons of ordinary skill in the art and includes, without limitation, desktop computers, laptop computers, tablets, servers, mainframe computers, smartphones, handheld computing devices, and the like.

In this disclosure, references are made to users performing certain steps or carrying out certain actions with their client computing devices/platforms. In general, such users and their computing devices are conceptually interchangeable. Therefore, it is to be understood that where an action is shown or described as being performed by a user, in various implementations and/or circumstances the action may be performed entirely by the user's computing device or by the user, using their computing device to a greater or lesser extent (e.g. a user may type out a response or input an action, or may choose from preselected responses or actions generated by the computing device). Similarly, where an action is shown or described as being carried out by a computing device, the action may be performed autonomously by that computing device or with more or less user input, in various circumstances and implementations.

In this disclosure, various implementations of a computer system architecture are possible, including, for instance, thin client (computing device for display and data entry) with fat server (cloud for app software, processing, and database), fat client (app software, processing, and display) with thin server (database), edge-fog-cloud computing, and other possible architectural implementations known in the art.

Generally, embodiments of the present disclosure are directed towards novel methods, devices, and systems that maintain or reduce internal lens pressure, including intralenticular pressure (i.e., pressure inside the capsule of the lens). In particular, the present disclosure relates to (1) reducing the presence and/or formation of proteins responsible for crowding, compacting, and/or causing increased internal lens pressure, and/or (2) maintaining circulating flux of ions into and out of the lens (e.g., reducing accumulation of calcium).

Lens Aggregation Diseases

Generally, lens aggregation diseases (e.g., presbyopia) manifest clinically in middle age. However, the processes that lead to presbyopia result in a dramatic increase in lens stiffness, which occurs well before the clinical onset of symptoms.

This onset of symptoms is commonly attributed, at least in part, to the loss of natural enzymatic and antioxidant protection in the eye against ultraviolet radiation (e.g., ultraviolet A (UV-A) and ultraviolet B (UV-B) radiation), with a concurrent increase in the production of photochemically active chromophores or oxidants over a period of time.

One skilled in the art will recognize that, as the lens of the eye absorbs light, chromophores are photoactivated and produce reactive oxygen species (e.g., singlet oxygen and superoxide). These oxidants denature lens proteins. At the same time as the lens accumulates such oxidants and/or oxidized components, there is a decreased efficiency of naturally-occurring mechanisms which repair proteins damaged by oxidation.

As a result, oxidative damage can cause progressive hardening of the lens substance, eventually reaching a level where the lens loses its ability to bend in order to focus, the focusing occurring through a process known in the art, and referred to herein, as “accommodation,” and the gradual stiffening causing the loss of ability to bend, which happens approximately between the ages of 42 and continues to around age 57 (known in the art, and referred to herein, as “presbyopia”). Gradual opacification of the lens results in a decreased amount of available light to the retina, thereby resulting in decreasing vision and/or cataract. This occurs, on average, between the ages of 65 and 75. Cataract, if left untreated, will result in surgically reversible blindness.

The cause of various lens aggregation diseases (e.g., presbyopia and cataractogenesis) is believed to be multi-factorial, and influenced by a combination of endogenous oxidation and exogenous oxidation. “Endogenous oxidation” means oxidation that occurs via internal mechanisms such as, for example, intraocular photochemical generation of superoxide and its derivatization to other oxidants such as singlet oxygen, hydroxyl radical, hydrogen peroxide and glycation, loss of the lens' and corneas' natural ultraviolet (UV) filters, and decreasing amounts of natural lens antioxidants, (e.g., glutathione). “Exogenous oxidation” means oxidation due to external and/or environmental factors, such as, for instance, increased exposure to short wavelength and UV radiation, ingestion of chemicals and pollutants, smoking, diseases such as diabetes, and the like.

It is commonly believed in the art that many types of oxidation, including those mentioned previously herein, denature healthy proteins, leading to a gradual loss of lens elasticity, which results in difficulty focusing at close distances (e.g., as occurs in presbyopia) and/or lens opacification (e.g., as results from cataracts).

However, lens aggregation diseases may not be solely, or mainly, initiated by oxidation; thus, such oxidation may not be the root cause of these diseases. While endogenous and exogenous oxidation are factors, other factors that are not recognized and/or under-recognized in the art include the effects on proteins from rising internal lens pressure.

For example, optical clarity of the lens is generally maintained through the process of normal protein folding and unfolding with respect to lens proteins. Healthy proteins fold, unfold, and refold with the help of so-called “molecular chaperones,” a term known in the art for proteins that assist in healthy folding and/or unfolding. Proteins may misfold and/or unfold due to a variety of factors, such as, for instance, oxidation, declining amounts of chaperones, and other factors. Misfolding may lead to protein aggregation, which, with respect to lens aggregation diseases, gradually hardens and opacifies the lens, thereby causing and/or exacerbating such diseases (e.g., presbyopia, cataracts).

Thus, conventional solutions have failed to adequately consider factors and/or changes other than exogenous and endogenous oxidation that may contribute to the onset and/or development of lens aggregation diseases.

Lens Pressure

Purely as a non-limiting example, changes in internal lens pressure may contribute to lens protein misfolding and/or unfolding, thereby leading to a loss of lens flexibility and the concomitant reduction in ability of the lens to focus.

It should be appreciated that, once any cellular protein is folded, various stress conditions may pose a threat to the protein's integrity. For instance, temperature variations, pressure, osmotic changes, antibiotics, solvents, and other chemicals and/or forces not only interfere with transcription, translation, and protein folding, but can also often disrupt the accurate three-dimensional protein structure.

It is generally known that pressure affects proteins, and more specifically, that pressure can unfold proteins. See, e.g., P. W. Bridgman, “The coagulation of albumin by pressure,” J Biol. Chem. 19:511-12 (1914); W. Kauzmann, “Thermodynamics of unfolding,” Nature 325:763-64 (1987).

Specifically, with respect to lens aggregation diseases, pressure may lead to precipitation and aggregation of proteins (e.g., lens proteins), cross-linking via disulfide bonds, reduction of glutathione, phosphorylation, and other post-translational protein modification factors that may cause progression of presbyopia and cataract.

Increased pressure may cause proteins to be penetrated by water in the same way they are penetrated by chemical agents (e.g., urea and guanidine) when such agents are added at high concentrations at atmospheric pressure. For instance, it is known that hyperbaric oxygen in vivo accelerates aging in the nuclear region of the guinea pig lens with regard to the loss of water-soluble and cytoskeletal proteins, damage to plasma membranes, formation of protein disulfide, and degradation of lens membrane protein MIP26. Such modifications are similar to those that occur in the nuclei of aging and cataractous human lenses.

In many cases, pressure drives proteins to unfold or misfold, resulting in intermediate, partially-folded protein conformations and molten-globular intermediates. Misfolded proteins, aggregates, and amyloids are usually derived from partially folded intermediates. Additionally, as compared to temperature, which affects a biological system's energy content, increased pressure generally shifts the equilibrium between associated and dissociated forms of proteins toward dissociation without affecting the system's energy content. Such increased pressure induces changes that range from small conformational effects, compressibility effects, and changes in populations of intermediate states to the complete loss of native folding of one or more proteins.

Additionally, in contrast to the low sensitivity of nucleic acids to pressure, lipid membranes are extremely sensitive to pressure, more so than water-soluble proteins. Alterations to lens membranes, including potentially as a result of oxidative processes, may contribute to development of lens aggregation diseases (e.g., cataracts). It is known that protein oxidation in lenses can initiate at lens membranes, and products of lipid oxidation in lenses can increase with both age and cataract development. It is further known that membrane derangement occurs in cataractous lenses in humans, potentially indicating that lipid oxidation and/or compositional changes in the membrane may cause lens opacification.

Various sources may cause such pressure inside the lens, leading to elevated internal lens pressure, as described in further detail below.

Hydrostatic Pressure

The space between the fiber cells of the lens is known as the interstitium. Fluid within the interstitium is termed the interstitial fluid. Such fluid circulates through the lens and fiber cells of the lens are accordingly bathed by, and within, the interstitial fluid. An intracellular gradient of hydrostatic pressure drives fluid from central fiber cells outward towards surface epithelial cells, pushing outwards against the lens capsule.

Mathematically, hydrostatic pressure is defined as a change in volume divided by a change in pressure. As a result, the more fluid that filters into the interstitium, the greater the volume of the interstitial space (Vᵢ) and the hydrostatic pressure within that space (Pᵢ). For instance, in young, healthy human lenses, an intracellular hydrostatic pressure gradient is from ˜340 mmHg in central fiber cells to ˜0 mmHg in surface cells.

Intralenticular (that is, located within the lens of the eye) hydrostatic pressure generally increases with age. Generally, as a consequence of accommodation, there is a tendency for water to move from the lens to the surrounding area(s), which then increase the osmolarity of the lens. The ratio of free water to bound water decreases with increasing pressure, and accordingly increases with decreasing pressure.

Accommodation often declines between the ages of forty and sixty. In the oldest normal human lenses, an increase in osmotic pressure causes the release of bound water to become free water. When such a response to pressure is irreversible, the released free water accumulates in so-called lakes.

This release of bound water from the hydration layers of macromolecules and its conversion to free water in condensed systems is known as syneresis, which is known as the extraction or expulsion of a liquid from a gel. In the lens, decreasing osmotic pressure induces syneresis.

During accommodation, liquid is expelled from its bound state with lens crystallins, thereby becoming free water, thus decreasing osmotic pressure. As the ability to accommodate is lost, the free water-to-bound water ratio decreases with increasing pressure, resulting in a significant syneretic response. The ability of the human lens to respond reversibly to pressure decreases with a decrease in accommodation. When the accommodation ability is lost altogether, an increase in free water, which may be a source of cataract formation, may ensue.

Younger lenses convert free water to bound water efficiently with increasing hydrostatic pressure, but, in older lenses, this ability is diminished and, in some cases, reversed. Generally, the total water content is much higher in cataractous lenses (that is, lenses affected by cataract) than normal lenses. Thus, in complete presbyopia (i.e., where there is no accommodation), the lens is fixed in its unaccommodated, compressed configuration, with a lower tendency for water movement out of the interstitium, thereby creating higher internal pressure. Therefore, with aging, the ability of the lens to compensate for increased hydrostatic pressure is decreased.

Compression

Another factor that may result in elevated internal lens pressure is compression that results via physical constraint of the lens.

Hydrostatic pressure affects other organs in the body besides the eye, including, for instance, the brain (which is encased by rigid bone) and the kidney (which is encased by a capsule). The lens, like the kidney, is confined by a capsule, and exists in a state in which small increases in fluid volume leads to large increases in pressure. Large increases in the interstitial pressure of tissue can lead to tissue damage and cellular death. Accordingly, constraint of the capsule surrounding the lens may add to increasing internal lens pressure.

In primates, the lens capsule itself is generally a strong, transparent membrane that is capable of shaping the lens and its surface curvature by participating in the process of accommodation. The capsule is an uninterrupted basement membrane completely enclosing the lens, sequestering the lens from other ocular tissues, protecting its optical integrity from penetration by large molecules and protecting the lens from infectious microbes (e.g., viruses, bacteria).

As the lens is avascular, the capsule must also allow for the passive exchange of metabolic substrates and waste in and out of the lens.

The elastic modulus of the capsule must therefore be sufficiently higher than that of the lens substance in order to allow the forces applied by the ciliary muscles to mold the lens shape. The adult human lens capsule has an elastic modulus of approximately two thousand times higher than the cellular lens cortex and nucleus that it surrounds.

During accommodation, the zonules, which insert into the lens capsule, apply stress that has both parallel (e.g., stretching) and perpendicular (e.g., compressive) components. These discrete stresses are transformed by the capsule into a uniform stress that is approximately perpendicular to the lens surface. The transition from the unaccommodated to the accommodated state would include a reduction of stresses perpendicular to the lens surface.

Under uniaxial load, capsular elastic moduli at 10% strain increase with age until about age thirty-five, from around 0.3 N/mm² to 2.3 N/mm², and then becomes relatively constant thereafter. In other words, past age thirty-five, the capsule load is maximized at around 2.3 N/mm².

On top of this, continual production of lens cell fibers by the lens epithelium in the environment constrained by the lens capsule, which is fixed in volume, accordingly contributes to continual crowding and compaction. The fluidic changes combined with continued pressure from the production of proteins in a confined space, result in increasing hydrostatic pressure with age and a syneretic process that continually increases resistance within the lens. This, in turn, leads to increased light scattering and a less pliable lens, decreasing the ability of the lens to accommodate, as seen in, e.g., presbyopia and other diseases.

Over time, there is an increase in lens stiffness and elastic modulus observed in the lens nucleus and cortex, resulting from the continual accession of fiber cells. As the elastic modulus of the lens substance increases, more force must be transmitted through the lens capsule to mold its shape. The inability of the lens capsule to achieve a sufficient elastic modulus over the lens substance in order to transmit the necessary forces for accommodation may be a key cause of presbyopia.

Effects of Pressure on Cellular Structure and Proteins

Pressure has also been shown to accelerate age-related loss of specific nuclear cytoskeletal proteins when compared to levels present in age-matched controls. These proteins include, for instance, actin, vimentin, ankyrin, alpha-actinin, and tubulin. Disulfide-crosslinking appears to be a primary cause of cytoskeletal protein loss. Additionally, pressure has been implicated as a cause of the disulfide cross-linking of MIP26 and other cytoskeletal proteins implicated in cataract development.

The effects of pressure may trigger the oxidative damage of gap junctions, which are clusters of intracellular channels that, when gated open, mediate the direct cell-to-cell transfer of low molecular mass (e.g., <1 kilodalton (kD)) substances including, for example, ions, secondary messengers, and nutritional metabolites. Since the lens lacks blood vessels, the lens must use non-vascular mechanisms to move nutrients into, and waste products out of, the fiber cells located in the center of the lens. This is achieved, in part, by an extensive network of gap junction channels that join the cells of the lens into what has been termed in the art as an ionic and metabolic syncytium. Indeed, mature fiber cells possess an exceptionally large number of gap junctions, likely the highest concentration in any tissue in the body.

The lens grows throughout life, so there is an age-dependent increase in lens size that impacts lens circulation. Increased size causes increases in intracellular gradients for, e.g., sodium, calcium, voltage, and hydrostatic pressure. Aging results in the accumulation of oxidative damage to membrane transport proteins, particularly fiber cell gap junction channels, which affect factors involved with lens circulation. Thus, aging has major effects on lens transport and homeostasis.

Maintaining and/or Reducing Lens Pressure

Accordingly, embodiments of the present disclosure are directed to novel methods, devices, and systems that reduce the formation of proteins responsible for crowding, compacting, and/or causing increased internal lens pressure. Without wishing to be bound by theory, a reduction in internal lens pressure may protect the lens from forming post-translational modifications, thereby treating (e.g., retarding, eliminating, or reversing) presbyopia and/or cataract.

The aforementioned may be achieved by, for example, genetically modifying cellular machinery that produces one or more of these proteins. Such machinery is located in the same portion of the lens epithelium mentioned above herein, that is, the germinative zone located between the lens anterior epithelial mid-periphery and the lens equator.

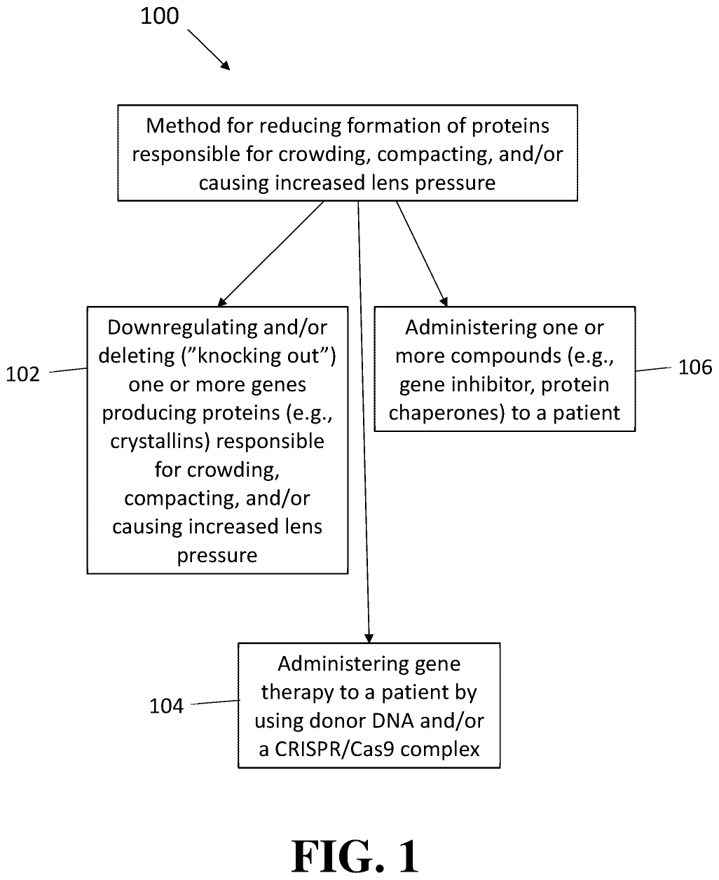

Turning now to FIG. 1, a method 100 is shown for reducing formation of proteins responsible for crowding, compacting, and/or causing increased lens pressure. The method 100 may comprise one or more of the steps shown at blocks 102, 104, and 106 in any combination.

The method comprises, at block 102, using one or more gene editing methods to downregulate and/or delete (also known as “knock out”) one or more genes, or combinations thereof, that produce proteins responsible for crowding, compacting, and/or causing increased internal lens pressure.

It should be appreciated that gene knock-out or deletion to reduce production of one or more proteins and/or crystallins (e.g., αB, βA4, βα3/α3, ββ2, and ββ2P1) can result in a reduction of internal lens pressure and enhancement of autophagy.

It should be noted, however, that not all crystallins are valid targets for gene deletion or knock-out, since these crystallins (including, for instance, αAβα2, Bα3, βα4, ββ1, ββ3, and all γ crystallins) serve a protective role against lens damage, loss of lens function, and/or cataract.

In at least one example, the production of lens crystallin proteins is reduced. These proteins include, but are not limited to, crystallin proteins produced by the germinative region of the anterior epithelium of the mammalian lens.

The one or more gene editing processes used may include, but not limited to, CRISPR (clustered regularly interspaced short palindromic repeats), TALENs (transcription activator-like effector nucleases), RLR (Retron Library Recombineering), ZFNs (Zinc finger nucleases), short synthetic single-stranded oligonucleotide modification (e.g., modification of ribonucleic acid (RNA)), nucleofection, and the like.

Additionally, various genes may be deleted or downregulated, including, for instance, the epithelial αB lens crystallin promoter gene, the epithelial βA4 lens crystallin promoter gene, the epithelial βα3/α3 lens crystallin promoter gene, the epithelial ββ2 lens crystallin promoter gene, the epithelial ββ2P1 lens crystallin promoter gene, the p38 mitogen-activated protein kinase (MAP kinase or MAPK) gene, and/or the Crystallin-50 (Cx50) gene.

The method 100 may further comprise, at block 104, administering gene therapy to a patient with one or more lens conditions (e.g., presbyopia, cataract, glaucoma, accumulation and/or aggregation of misfolded proteins in the lens, loss of clearance of misfolded proteins in the lens, oxidation of misfolded proteins in the lens, decrease in lysosomal activity in the lens, cell death in the lens) or symptoms thereof. The gene therapy may comprise donor deoxyribonucleic acid (DNA) and/or a CRISPR/Cas9 complex to deliver the donor DNA and/or the CRISPR/Cas9 complex inside the cells of the patient.

The method 100 may additionally comprise, at block 106, administering one or more compounds to the patient. The one or more compounds may include an inhibitor that reduces, inhibits, and/or eliminates expression of one or more of the genes described above herein. The inhibitor may include, for instance, a gene knockdown construct and/or artificial nucleic acid molecule (e.g., small interfering RNA (siRNA), microRNA (miRNA), short hairpin RNA (shRNA), antisense RNA, single guide RNA (sgRNA)), and/or a gene editing nuclease knockdown (e.g., via CRISPR/Cas9) of activating transcription factor 4 (ATF4) and/or C/EBP homologous protein (CHOP). In at least one example, the aforementioned knockdown construct targets an exon, intron, or exon/intron junction of one or more of the genes described above herein.

In at least an additional example, the one or more compounds may also include synergistic amounts of one or more protein chaperones to assist in proper protein folding. Non-limiting examples of such chaperones include an alpha-crystallin protein, a beta-crystallin protein, a gamma crystallin protein, and one or more proteins from within the heat shock proteins (Hsps) of molecular chaperones.

The one or more compounds may further include one or more pharmaceutically acceptable excipients known in the art.

The one or more compounds and/or methods described above may be administered via a variety of routes, including, for instance, subcutaneous, cutaneous, intravitreal, intraocular, and/or ocular.

Further, it should be appreciated that the one or more compounds and/or methods described herein may result in reduction of protein misfolding, reduction of endoplasmic reticulum stress, reduction of defective autophagy, and/or increase of autophagy.

Additional features of the aforementioned examples and embodiments are presented in the Examples below herein.

Gap Junction Coupling

The fibers in the center of the lens are uniquely dependent on intercellular communication with cells at the lens surface because they have no blood supply. Lens fibers have been shown to be joined into a syncytium with respect to ions. This syncytium is achieved by gap junctions between adjacent fibers, which permit ions and small transported metabolites to diffuse from cytoplasm to cytoplasm between adjacent cells.

Gap junctions themselves are composed of integral plasma membrane proteins known as connexins, three of which are present in the lenses of many species. The aforementioned age-related changes include the reduction in fiber cell gap junction coupling conductance, which can be caused by oxidative damage to lens connexins. Since fiber cell connexins are sensitive to oxidative damage and increases in lens size, the downregulation of gap junction coupling that occurs with age causes depolarization of the intracellular voltage and increases in intracellular concentrations of sodium and calcium. Depolarization and increased intracellular sodium concentration result in reductions of the transmembrane driving force of fiber cell membrane sodium-dependent transporters, which are also responsible for intracellular homeostasis. These effects compromise intracellular proteins and allow increased oxidative damage to crystallins and aggregation.

With age, the lens appears to compromise circulation in order to maintain a more constant pressure gradient, though such maintenance is imperfect. The age-dependent decline in the optical efficiency of the lens may be driven, at least in part, by changes in the lens circulation. Intracellular hydrostatic pressure in the lens is expected to vary in proportion according to the ratio of α²j_{Na}/Nⱼ, where a is the lens radius (in centimeters), j_{Na} is the average density of the fiber cell transmembrane influx of sodium (Na) (in moles per centimeter²), and Nⱼ is the number of open gap junction channels per area of fiber cell-to-cell contact (in centimeters⁻²).

Based on the above, and knowing that the number of open gap junction channels per area of fiber cell-to-cell contact decreases with age, it is possible to calculate that the hydrostatic pressure in the lens increases with age.

For example, it is known that, in lenses in which gap junction coupling is increased, the central pressure is lower. See, e.g., J. Gao et al., “The Effects of Age on Lens Transport,” Invest. Ophthalmol. Vis. Sci. 2013, Vol. 54, pages 7174-7187. However, if gap junction coupling is reduced, the central pressure is higher but the surface pressure is always zero. See, e.g., id. Thus, reduction in gap junction coupling is a direct cause of the age-related increase in intracellular hydrostatic pressure in the lens nucleus.

As coupling conductance decreases, forces that drive lens circulation must increase in order to maintain the circulating fluxes. For example, there must be increases in the intracellular hydrostatic pressure gradient, in the diffusion gradient for sodium, and in the voltage gradient. Both the accumulation of intracellular sodium and the depolarization of the intracellular voltage due to age leads to a reduction in the fiber cell transmembrane electrochemical potential for sodium entry. Consequently, sodium influx decreases and, since this drives water flow, water flow is also decreased, thereby moderating the increase in gradients for, e.g., intracellular sodium concentration, voltage, and hydrostatic pressure. Additionally, hydraulic conductivity depends on the number of open gap junction channels. Thus, both reduced hydraulic conductivity and increased radius of the lens cause the intracellular hydrostatic pressure (known as pᵢ) to increase with age.

Coupling is also essential for fiber cell homeostasis, since uncoupled mature fibers (MF) depolarize and subsequently become opaque. This increase in hydrostatic pressure, in addition to decreased coupling conduction, is an initiator of the oxidative processes described above herein that lead to lens degeneration diseases (e.g., presbyopia and cataract). Gap junctions in the MF survive for the lifetime of the organism without protein turnover.

It is known that α3 connexin has a role in coupling MF to peripheral cells. It is possible that α3 connexin provides long-term communication in mature fibers; thus, α3 connexin may help maintain lens transparency. For instance, lenses of so-called “knock-in” α3 mice, where α3 connexin is expressed under the endogenous α8 connexin locus, are transparent but smaller.

Cleavage of connexins occurs abruptly between the peripheral shell of differentiating fibers (DF) and the inner core of MF. The appearance of the cleaved connexins is generally correlated to a change in coupling conductance. The DF remains coupled, but conductance is reduced to 30 to 35% of normal. However, the gap junctions in the DF of α3 negative/negative lenses remained sensitive to pH. It is possible that there is active transport by peripheral cells to maintain a homeostatic environment for cells in the MF zone. Loss of coupling would cut off the MF zone from this circulation.

Thus, embodiments of the disclosure prevent this loss of coupling and the attendant cutoff of the MF zone from this circulation. Without wishing to be bound by theory, cutting off the MF zone from circulation causes changes in glutathione levels that occur around age 40 and can lead to cataract.

Further embodiments reduce intralenticular pressure by the upregulation of gap junction channels, thereby evening out osmotic balance and reducing the disruption of lens fibers from syneretic processes.

Additional embodiments disclosed herein manipulate gap junction proteins, pathways that phosphorylate these gap junction proteins, and/or proteases (e.g., calpain) that destroy gap junction proteins. This results in maintaining the osmotic gradient and lens calcium levels, thereby preventing increasing lens hydrostatic pressure by maintaining a healthy circulation of water in and out of the lens.

Role of Calcium

The reduction in gap-junction coupling mentioned above herein results in the increased retention of calcium, which causes an osmotic imbalance that leads to an inflow of water into spaces in-between the lens fibers. This can then contribute to the onset of presbyopia and/or cataract.

At the lens surface, calcium (Ca)-adenosine triphosphatase (Ca-ATPase) and sodium (Na)/Ca exchange occurs. This sets up circulation of Ca²⁺, where the path to the lens surface depends on the presence of gap junctions.

Multiple plasma membrane Ca²⁺-ATPase isoforms are expressed from four PMCA (plasma membrane calcium ATPase) genes (specifically, PMCA1-4) and alternative messenger RNA (mRNA) splicing. All four PMCA genes are expressed in the lens epithelium, the PMCA3 transcript being the most abundant. The transcripts for PMCA1, PMCA2, and PMCA4 also exist in decreasing order of abundance. PMCA assists in the extrusion of calcium from cells.

Gap junction coupling of interior fiber cells to the surface cells is an essential component of Ca homeostasis. The MF deep within the lens do not have Ca-ATPase activity or Na/Ca exchange to transport calcium out, yet have membranes that are permeable to calcium. Hence, calcium is continuously leaking into these cells throughout the volume of the lens. Ca²⁺ accumulates in MF until its diffusion to surface balances the equilibrium.

Loss of coupling cuts off the MF zone from this circulation. With time, ion gradients in uncoupled cells dissipate, intracellular calcium increases, proteolysis of cytoplasmic proteins (such as one or more crystallins) occurs, and denatured proteins aggregate and form light-scattering elements that may be responsible for nuclear opacity. Indeed, it is known in the art that elevated lens calcium and cataract are correlated, and calcium is also known to produce aggregation of proteins when added to solutions of soluble lens proteins.

Further, a progressive loss in cytoskeletal proteins is correlated with an increase in free calcium, causing most spectrin and vimentin (both of which are types of structural and/or cytoskeletal proteins) present in the lens cortex to disappear. This indicates that proteolysis by calcium-dependent enzymes (e.g., calpain) may play a significant role in cytoskeletal regulation and metabolism in the lens.

Calcium-induced transparency loss has at least two phases. At moderately increased calcium levels, opacification occurs without major degradation of various intracellular proteins and may be the result of calcium-stimulated interactions between the membrane-cytoskeletal network and one or more crystallins. Such a calcium-induced interaction between structural elements of the lens could be reversible.

Role of Cyclic Adenosine Monophosphate (cAMP)

Intracellular levels of cAMP are regulated by the balance between the activities of two enzymes, adenylyl cyclase (AC) and cyclic nucleotide phosphodiesterase (PDE). cAMP can bind to, and modulate the function of, a family of cyclic-nucleotide-gated ion channels. These are relatively non-selective cation channels that conduct calcium. Calcium stimulates calmodulin (CaM) and CaM-dependent kinases and, in turn, modulates cAMP production by regulating the activity of ACs and PDEs.

The CREM (cAMP responsive element modulator) gene, which encodes for the cAMP responsive element modulator protein, is also known to encode the powerful repressor also encodes the powerful repressor ICER (inducible cAMP early repressor), which negatively feeds back on cAMP-induced transcription.

Role of Ligands that Enhance Gap Junction Communication

Ligand-gated ion channels (referred to alternatively as “LIC” or “LGIC”), also known as ionotropic receptors, are transmembrane ion-channel proteins that open. By opening, the LICs allow ions (e.g., Na⁺, K⁺, Ca²⁺, and/or Cl⁻) to pass through the membrane in response to the binding of a chemical messenger (e.g., a ligand such as, for instance, a neurotransmitter). The LIC superfamily includes, for example, nicotinic acetylcholine receptors (nAChRs), adenosine triphosphate (ATP) receptors, γ-aminobutyric acid (GABA) receptors, glutamate receptors, glycine receptors, and 5-hydroxytryptamine (5-HT) receptors.

Various ligands that may enhance gap junction communication include, for instance, aquaporins, protein kinase C (PKC), filensin, pigment epithelium-derived factor (PEDF), and fibroblast growth factor (FGF), each of which will be described briefly below.

Lens epithelial cells express various aquaporins (e.g., aquaporin-1 (AQP1), aquaporin-5 (AQP5), and aquaporin-7 (AQP7)). AQP5 and AQP7 are expressed in lesser amounts than AQP1. Fiber cells also express, e.g., aquaporin-0 (AQP0) and AQP5, the latter of which is expressed in lesser amounts. AQP1 is the main aquaporin (AQP), representing a majority of AQPs in human lymphatic endothelial cells (HLEC). Further, AQP1 protein expression appears to be increased in HLEC from cataract patients. AQP5 protein expression may also similarly be increased. As a result, the regulation of AQP1 and/or AQP5 can help maintain lens transparency.

PKC positively regulates both water permeability and ionic conductance of AQP1 channels. Activation of PKC results in the phosphorylation of cellular proteins. Stimulation of PKC is dependent upon diacylglycerol (DAG) signal. PKCs are regulated by a variety of lipid secondary messengers, including diacylglycerol and phosphatidylserine. Further, phosphorylation of the enzyme plays a critical role in its activation.

The filensin and PEDF genes are known in the art to have important roles in the physiology and morphology of the transparent lens. Filensin is an intermediate filament protein and a component of the filament of lens fiber cells, while PEDF is a multifunctional secreted protein. Downregulation of the expression of the filensin and/or PEDF genes may therefore contribute to the formation of cataract.

FGF signaling is required for upregulation of gap junction mediated intercellular coupling (GJIC) at the lens equator. The mammalian FGF receptor (FGFR) family consists of four genes, specifically FGFR1 through FGFR4. Varieties of FGFR1 through FGFR4 proteins are produced that have differing affinities for ligands. Further, FGF has the ability to increase GJIC in cultured lens cells.

Further, gene modification may be used to regulate water, sodium, and/or calcium ion transport and storage of sarco-endoplasmic reticulum gating and lens fiber cell channels, thereby reducing or eliminating oxidative damage of gap junctions by upregulating gap junction coupling. Without wishing to be bound by theory, the aforementioned may occur via sustained activation of Extracellular Signal-Regulated Kinase (ERK).

Accordingly, without wishing to be bound by theory, an increase in internal lens hydrostatic pressure can result in several cascading effects, including, for instance, oxidation, connexin damage (e.g., to Cx43, Cx46, and/or Cx50), gap junction failure, a loss of lens circulation, calcium accumulation, and, finally, presbyopia and/or cataract.

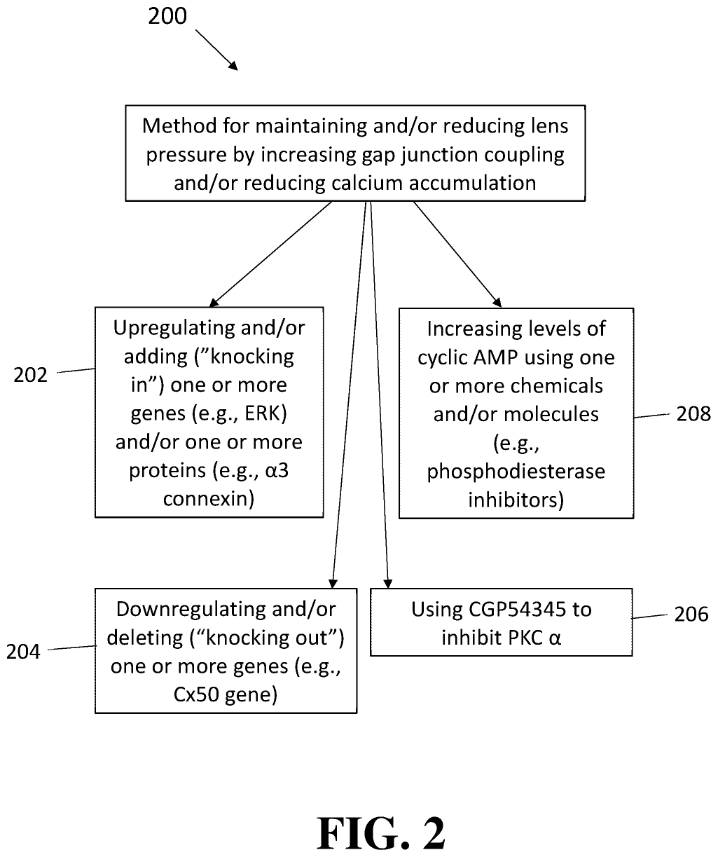

Turning now to FIG. 2, a method 200 is shown for maintaining and/or reducing internal lens hydrostatic pressure by increasing gap junction coupling and/or reducing calcium from accumulating within the lens. The method may boost proteins and enzymes that (1) form and protect gap junctions, and (2) increase gap junction coupling. It should be appreciated that the method 200 may comprise one or more of the steps shown at blocks 202, 204, 206, and 208 in any combination.

The method 200 comprises, at block 202, genetically upregulating and/or adding (also known as “knocking in”) one or more proteins and/or one or more genes. The one or more proteins may include proteins that are protective, or enhance the function, of FGF, such as, for instance, extracellular signal-regulated kinase (ERK), aquaporin-0, one or more PKC isotypes (including the PKC γ isotype), calpastatin, phenylmethylsulfonyl fluoride (PMSF), and/or leupeptin. Additional proteins that may be knocked-in include, for example, α3 connexin.

The one or more genes may include one or more genes, or portions thereof, that express one or more of the aforementioned proteins. Additional genes that may be knocked in include, for example, ERK, protein kinase A (PKA), cyclic AMP (cAMP), PMCA1, PMCA2, PMCA3, PMCA4, FGFR1, FGFR2, FGFR3, FGFR4, genes for aquaporin-0, aquaporin-1, and/or aquaporin-5, genes for PMSF, genes for leupeptin, a gene expressing the inhibitor compound BIRB796, genes expressing filensin (including a 50 kDa form of filensin), phakinin, inwardly rectifying potassium channel (IRPC) proteins, and/or PEDF, genes expressing CaM-dependent phosphodiesterase, genes for adenylate cyclase, the PDE gene(s), and/or the genes for calsequestrin, triadin, and/or junctin.

Further knock-in candidates include, for example, one or more genes to upregulate expression levels of SERCA (sarcoplasmic/endoplasmic reticulum calcium ATPase) and/or PMCA pumps (e.g., PMCA1, PMCA2, PMCA3, and/or PMCA4), thereby increasing calcium concentration (e.g., to a physiological resting level).

The method 200 may further comprise, at block 204, downregulating and/or knocking-out one or more genes, such as, for example, the Cx50 gene. Deletion of Cx50 may occur either with, or without, addition of the Cx46 gene. Additional genes that may be downregulated and/or knocked out include, for instance, CREM, PKC 7, a gene expressing MicroRNA-182-5p, the calmodulin gene, genes for nitric oxide synthetase, genes for inositol 1,4,5-trisphosphate 3-kinases (IP3K), genes for dystroglycan (DAG), genes for protein kinase A, genes for protein kinase C, genes for phospholipase C (PLC), genes for cAMP-dependent phosphodiesterase, the G-protein gene, and/or the decorin (DCN) gene. It should be appreciated that gene knock-outs may be done using any process known in the art, including, for instance, alternative mRNA splicing.

The method 200 may further comprise, at block 206, using CGP54345 to specifically inhibit PKC α.

The method 200 may additionally comprise, at block 208, increasing levels of cAMP using one or more chemicals and/or molecules (e.g., phosphodiesterase (PDE) inhibitors, forskolin (also known in the art as coleonol), and/or 8-bromo-cyclic AMP). Delivery of one or more PDE inhibitors may be achieved via one or more viral vectors and/or one or more monoclonal antibody delivery systems. Non-limiting examples of PDE inhibitors include: 3-isobutyl-1-methylxanthine (also known in the art as IBMX), pentoxifylline, propentofylline, ibudilast, A23187 (also known in the art as Calcimycin), beta-adrenergic agents and/or agonists (e.g., epinephrine, norepinephrine, and isoproterenol), salbutamol, vasoactive intestinal peptide (VIP), dibutyryl cyclic AMP (dB-cAMP), prostaglandin E2 and its analogs, prostaglandin I2 (also known in the art as prostacyclin) and its analogs, choleragen (CTX), cycloheximide, actinomycin D, dexamethasone, D-Limonene, SB 203580, fisetin, honokiol, epigallocatechin gallate, and grape seed proanthocyanidin.

Mitogen-Activated Protein Kinase and Crystallin Phosphorylation

Phosphorylation processes appear to play a role in the onset and/or progression of various lens aggregation diseases, including, for instance, cataracts. Phosphorylation in the lens epithelium may be, at least on some levels and at initial stages, reversible.

Mitochondria in the lens cortex removes most oxygen, thereby maintaining low oxygen tension in the lens nucleus. During oxidative phosphorylation, some electron leakage to oxygen occurs, forming superoxide. Increased mitochondrial production of superoxide occurs with age. This process contributes to elevated H₂O₂ in the nucleus of older lenses.

Additionally, phosphorylation is the most common post-translational modification of various crystallins (e.g., the α-crystallins) in the human lens. Indeed, phosphorylation of αB-crystallin can result in uncontrolled protein aggregation.

It is known that crystallin αB, crystallin βA4, and phosphorylation and truncation of crystallins in the lens, all increase with the formation of lens opacity. Such increases may contribute to the formation of cataracts. Moreover, phosphorylation and truncation of these proteins increased with the progression of cataracts.

Some of the major protein modifications include, for example, phosphorylation of αB-crystallin at serine (Ser) residues 19, 45, and 59. In particular, the phosphorylation of αB-crystallin at Ser-45 results in uncontrolled aggregation.

Phosphorylation of a-crystallin subunits also occurs, and is likely catalyzed by a specific mitogen-activated protein kinases (MAP kinases or MAPKs), namely, MAP kinase-activated protein kinase (MAPKAP kinase)-2. MAP kinases, which regulate cellular properties in response to a wide range of extracellular stimuli, are known to phosphorylate the OH group of serine (Ser) or threonine (Thr) in proteins and play important roles in the regulation of cell proliferation, differentiation, survival, and apoptosis.

Additionally, there is increased localization of phosphorylated αB-crystallin to the cytoskeleton. Chronic perturbation of the intermediate filament network, and/or disorganization and/or disruption of other cytoskeletal networks, can result in the activation of p38 MAP kinase. This activation can lead to the aforementioned Ser-59 phosphorylation of αB-crystallin and its colocalization with cytoskeletal elements.

Ser-45 phosphorylation of αB-crystallin is mostly involved in the cell cycle, while Ser-59 phosphorylation of αB-crystallin prominently occurs under stress conditions. Thus, without wishing to be bound by theory, targeting one or more stress- or inflammation-related kinase pathways (e.g., the p38 MAP kinase pathway and the p44 kinase pathway) could result in the moderation of phosphorylation of αB-crystallin.

There is currently no known inhibitor for MAPKAP kinase-2 (which is a substrate of the p38 MAPK) that phosphorylates αB-crystallin. However, inhibition of either p38 MAPK or its activation (upstream to p38 MAPK) could be beneficial. Such inhibitors of p38 MAPK have been developed to target the kinase in the context of treating some human diseases (e.g., arthritis, cancers involving inflammation). Additionally, transient and/or temporary inhibition of the p38 MAP kinase pathway could also be beneficial, since low levels of phosphorylation of αB-crystallin may have protective effects. Such transient and/or temporary inhibition would also ameliorate the deleterious consequences, if any, of completely and/or irreversible inhibition of p38 MAPK.

Accordingly, embodiments of the present disclosure relate to methods for reducing internal lens hydrostatic pressure by decreasing phosphorylation of one or more crystallins (e.g., the αB-crystallin). In at least one example, decreased phosphorylation of αB-crystallin occurs via inhibition of one or more proteins and/or genes in the p38 MAPK pathway.

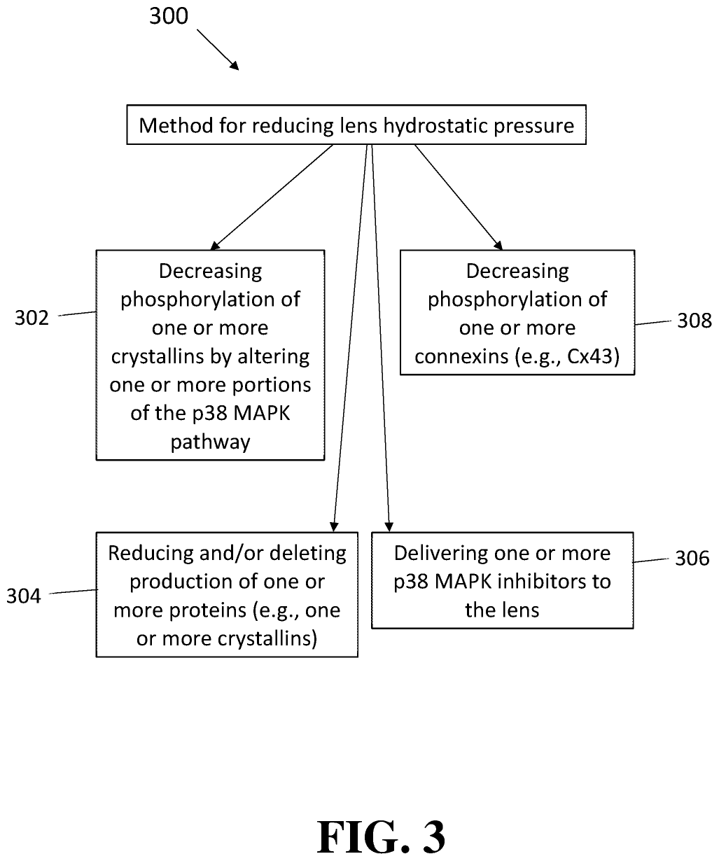

Turning now to FIG. 3, a method 300 is shown for reducing internal lens hydrostatic pressure. The method 300 may comprise one or more of the steps shown in blocks 302, 304, 306, and 308 in any combination.

The method 300 comprises, at block 302, decreasing phosphorylation of one or more crystallins by altering one or more portions of the p38 MAPK pathway. The one or more crystallins may include, for instance, αB-crystallin. The alteration of one or more portions of the p38 MAPK pathway may be achieved by, for example, knock out of one or more genes (e.g., MAPK11, MAPK12, MAPK13, MAPK14, p38 MAPK gene).

The method 300 may additionally comprise, at block 304, reducing and/or deleting production of one or more proteins (e.g., αB crystallin, βA4 crystallin, βα3/α3, ββ2, and ββ2P1).

The method 300 may further comprise, at block 306, delivering one or more p38 MAPK inhibitors to the lens. Delivery may be achieved by one or more processes known in the art, for example, viral vectors or monoclonal antibodies. Non-limiting examples of p38 MAPK inhibitors include SB 203580, SB 239063, SB239063AN, FR 167653, SB-681323 (also known in the art as dilmapimod), losmapimod, BIRB796, VX-702, ralimetinib (also known in the art as LY2228820 dimesylate), angiotensin 1-7, PHA 666859, SD-282 (ICS p38IH), SB202190, AZD7624, ML3403, PD169316, one or more vitamin E analogs (e.g., vitamin E analog 7), skepinone-L, LN950, CBS3830, VX-745 (also known in the art as neflamapimod), JLU1124, rituximab, RO4399247, AVE8677, compound 35, compound 36, compound 37, peptide 11R-p38I110, PH797804, prexasertib (also known in the art as LY2606368), ginsenoside Rg1, 6-hydroxy-2,5,7,8-tetramethylchroman-2-carboxylic acid (also known as Trolox), MW181, icarin, apigenin, astaxanthin, N-Benzylcinnamide, L-3-n-Butylphthalide, z-Ligustilide, (+)-2-(1-Hydroxyl-4-oxocyclohexyl) ethylcaffeate, macranthoin G, 4-O-methylhonokiol, L-Theanine, 3,4-Dihydroxyphenyl ethanol, linalool, pinocembrin, puerarin, tanshinone IIA, MW01-2-069A-SRM, obovatol, glaucocalyxin B, α-iso-cubebene, floridoside, NOSH-aspirin (also known in the art as NBS-1120), esculentoside A, triptolide, and skepinone-L. See, e.g., S. Schnyder-Candrian et al., “Dual Effects of p38 MAPK on TNF-Dependent Bronchoconstriction and TNF-Independent Neutrophil Recruitment in Lipopolysaccharide-Induced Acute Respiratory Distress Syndrome,” J. Immunol. 175(1): 262-269 (Jul. 1, 2005); and J. K. Lee et al., “Recent Advances in the Inhibition of p38 MAPK as a Potential Strategy for the Treatment of Alzheimer's Disease,” Molecules 22(8): 1287 (Aug. 2, 2017).

The method 300 may further comprise, at block 308, decreasing phosphorylation of one or more connexins, such as, for example, Cx43.

Inhibition of Calpain and/or Calpain II

Calpains are Ca²⁺-dependent cysteine proteases. The unregulated Ca²⁺-mediated proteolysis of essential lens proteins by calpains might contribute to lens degeneration diseases (e.g., some forms of cataract) in both animals and humans. Further, the molecular structures of calpains have provided details that can be used to design calpain inhibitors. Such inhibitors have the potential to act as anti-cataract agents.

Five calpains are known in the art to occur in the lens, including calpain 1 (μ-calpain), calpain 2 (m-calpain), calpain 10, Lp82, and Lp85.

Additionally, the breakdown of vimentin in the outer cortex of the human lens, which occurs during aging, may be due to calpain. Bovine lens calpain 2 has been shown to degrade α-crystallin, actin, and vimentin, suggesting potential involvement of calpain 2 in the aging process. Both phenylmethylsulfonyl fluoride (PMSF) and leupeptin have also been shown to decrease vimentin degradation.

Further, proteolysis of lens cytoskeletal proteins (e.g., spectrin and vimentin) correlate with the loss of lens transparency. Lens proteins undergoing limited proteolysis by calpain may no longer interact properly, resulting in opacity.

Calpain inhibitors have been shown to reduce the rate of cataract formation. For instance, calpain 3 is necessary for the formation of age-dependent nuclear cataracts in α3Cx46^{-/-} mice. The loss of α3Cx46 leads to increased levels of Ca²⁺ ions, which activates Lp82/Lp85, thereby leading to the formation of a nuclear cataract.

Calpastatin, a calpain-specific inhibitor, is the endogenous inhibitor of at least two calpains, calpain 1 and calpain 2, the latter of which cleaves actin and vimentin. Additional exogenous calpain inhibitors are also being designed that focus on active-site-targeted small peptide analogues, which can provide a high selectivity for one or more calpains, coupled with good cell permeability and low toxicity. Without wishing to be bound by theory, at least some of these active-site-targeted small peptide calpain inhibitors act by binding the sulfhydryl group of cysteine (Cys) within the calpain active site, thereby inactivating the calpain.

Further, reducing calcium induced post-translational modification of proteins may be achieved by downregulating lens epithelial cell calcium influx or upregulating calcium efflux through controlling activation of calpastatin and/or the calpain family of enzymes. The aforementioned may be achieved by gene modification techniques, including, for instance, inhibiting the SRC kinase-dependent signaling pathway, inhibiting the calpain family of enzymes, and/or applying atropine.

Additionally, since human lens muscarinic receptors (e.g., in the iris, ciliary muscles, and retina) remain functional throughout life, they are also potential targets for cataract-inducing compounds.

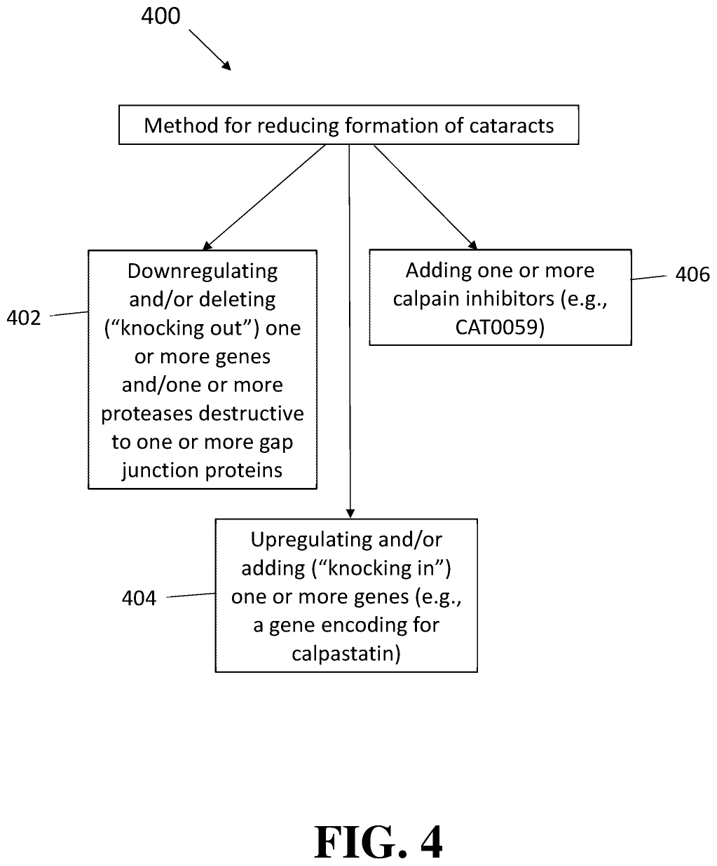

Turning now to FIG. 4, a method 400 is shown for reducing formation of cataracts. The method 400 may comprise one or more of the steps shown in blocks 402, 404, and 406 in any combination.

The method 400 comprises, at block 402, downregulating and/or deleting (“knocking out”) one or more genes and/or one or more proteases destructive to one or more gap junction proteins. The one or more genes that may be knocked out include, for instance, one or more genes encoding for one or more calpains (e.g., calpain 2, calpain 3, calpain-Lp82 (the isoform Lp82), calpain-Lp85 (the isoform Lp85), calpain 10, and/or Transglutaminase (TGase)), genes for calcium-dependent proteases, genes for m-calpains, genes encoding for enzyme proteases, genes for SRC kinase, and/or genes for myosin light-chain (MLC) kinase.

The method 400 may additionally comprise, at block 404, upregulating and/or adding (“knocking in”) one or more genes, such as, for instance, a gene for encoding calpastatin.

The method 400 may further comprise, at block 406, adding one or more calpain inhibitors. Non-limiting examples of such inhibitors include CAT0059 and/or one or more active-site-targeted small peptide calpain inhibitors that have an aldehyde C-terminal group (e.g., Cbz-Val-Phe-H, SJA6017, and the like).

Table 1 below displays a list of possible knock-out and/or deletion candidates mentioned in the present disclosure, along with specific single guide RNA (sgRNA) sequences for each candidate. PAM (protospacer adjacent motif) sequences are underlined. Predicted efficiency and the number of off targets is also displayed. Information from the table was generated using CHOPCHOP and the Benchling© platform.

TABLE 1
sgRNA sequences for knock-out and/or deletion candidates.

| Knock-out | sgRNA sequence | Predicted | Number of off |
|---|---|---|---|
| candidates | (PAM underlined) | efficiency | targets |
| CALM1 | CGTCACGTCATGACAAACTTAGG | 51% | 2 |
| (Calmodulin) | (SEQ ID NO. 43) | | |
| | GACGGACAAGTCAACTATGAAGG | 50% | 3 |
| | (SEQ ID NO. 44) | | |
| | GGCATTCCGAGTCTTTGACAAGG | 67% | 3 |
| | (SEQ ID NO. 45) | | |
| NOS1 | CGTATTCATCAGCCGGATGAGGG | 60% | 0 |
| (Nitric Oxide | (SEQ ID NO. 46) | | |
| Synthetase) | GGTCACTTCGTACGTTCGCAGGG | 60% | 0 |
| | (SEQ ID NO. 47) | | |
| | TGTCCGAAGGCGCAAAAGTGAGG | 60% | 0 |
| | (SEQ ID NO. 48) | | |
| MLCK | TCGCGATTTAGAAGTIGTGGAGG | 61% | 0 |
| (Myosin Light Chain | (SEQ ID NO. 49) | | |
| Kinase) | CGCGATTTAGAAGTIGTGGAGGG | 57% | 1 |
| | (SEQ ID NO. 50) | | |
| | CGTCCTCATCGTAGTCTATCTGG | 34% | 1 |
| | (SEQ ID NO. 51) | | |
| PI3KCA | GGATTTAGCTATTCCCACGCAGG | 57.15% | 1 |
| (Phosphatidylinositol | (SEQ ID NO. 52) | | |
| 3-kinase) | TAGCTATTCCCACGCAGGACTGG | 47.46% | 1 |
| | (SEQ ID NO. 53) | | |
| | TCGAATATTTACATTCACGTAGG | 61.64% | 0 |
| | (SEQ ID NO. 54) | | |
| CAPN1 | GGCAAATAGGTGAACTACCGAGG | 70.68% | 0 |
| (Calpain 1) | (SEQ ID NO. 55) | | |
| | AATGGCCATCCGCATCTCGTAGG | 62.31% | 1 |
| | (SEQ ID NO. 56) | | |
| | CGCGGTCTACGAGGTCAGGAGGG | 58.83% | 0 |
| | (SEQ ID NO. 57) | | |
| CAPN2 | AGCGGTTGAGCACCTCCCGGAGG | 70% | 2 |
| (Calpain 2) | (SEQ ID NO. 58) | | |
| | GTACTCGGTCACCGGAGCCGAGG | 70% | 3 |
| | (SEQ ID NO. 59) | | |
| | ACCGAGCAACGATGACTTGGTGG | 55% | 0 |
| | (SEQ ID NO. 60) | | |
| CAPN3 | GAGCGCAATCATGCTACGGCAGG | 52.14% | 0 |
| (Calpain 3) | (SEQ ID NO. 61) | | |
| (includes isoforms | TCGCATCTCGTAGCTGTTGATGG | 37.89% | 1 |
| Lp82 and Lp85) | (SEQ ID NO. 62) | | |
| | CCGTAGCATGATTGCGCTCATGG | 53.2% | 0 |
| | (SEQ ID NO. 63) | | |
| CAPN10 | GAGTTTGACGAGCTCACCGTTGG | 56% | 0 |
| (Calpain 10) | (SEQ ID NO. 64) | | |
| | CACCGTTGGCTACCCGGTCACGG | 57% | 1 |
| | (SEQ ID NO. 65) | | |
| | CTACGCCAAGTGCGTGTGCTGGG | 40% | 1 |
| | (SEQ ID NO. 66) | | |
| TGM2 | GTTGTAGTTGGTCACGACGCGGG | 65% | 0 |
| (Transglutaminase | (SEQ ID NO. 67) | | |
| (TGase) 2) | TGCGGAGGTCAATGCCGACGTGG | 63% | 0 |
| | (SEQ ID NO. 68) | | |
| | ACGCTGCCGATCCAGGACATGGG | 54% | 1 |
| | (SEQ ID NO. 69) | | |
| PRKCG | CACGAAGTCGGGGTTCACGTAGG | 65% | 0 |
| (Protein Kinase C | (SEQ ID NO. 70) | | |
| Gamma) | GAACCCCGACTTCGTGCACCCGG | 64% | 0 |
| | (SEQ ID NO. 71) | | |
| | TGACAAGTTCTTCACGCGGGCGG | 60% | 0 |
| | (SEQ ID NO. 72) | | |
| CAMP | GCGGTAGAGGTTAGCATCCGAGG | 68% | 2 |
| | (SEQ ID NO. 73) | | |
| | CTATAGATGGCATCAACCAGCGG | 72.82% | 4 |
| | (SEQ ID NO. 74) | | |
| | CAGCTGGTGAAGCGGTGTATGGG | 41.89% | 3 |
| | (SEQ ID NO. 75) | | |
| p38 | CACAAAAACGGGGTTACGTGTGG | 60% | 1 |
| | (SEQ ID NO. 76) | | |
| | AGCTCCTGCCGGTAGAACGTGGG | 60% | 2 |
| | (SEQ ID NO. 77) | | |
| | ATAATGGCCGAGCTGTTGACTGG | 58% | 2 |
| | (SEQ ID NO. 78) | | |
| Src Kinase | GCCGAGCCCAAGCTGTTCGGAGG | 60.33% | 2 |
| | (SEQ ID NO. 79) | | |
| | CGATCTGCAGGCGGAACCGTGGG | 58.94% | 2 |
| | (SEQ ID NO. 80) | | |
| | CTATGACTATGAGTCTAGGACGG | 55.36% | 2 |
| | (SEQ ID NO. 81) | | |
| MLCK/MYLK | TCGCGATTTAGAAGTTGTGGAGG | 61.19% | 0 |
| | (SEQ ID NO. 82) | | |
| | CGCGATTTAGAAGTIGTGGAGGG | 57.43% | 1 |
| | (SEQ ID NO. 83) | | |
| | CGTCCTCATCGTAGTCTATCTGG | 35.88% | 1 |
| | (SEQ ID NO. 84) | | |
| GJA8 | CCCCGTCCCTGATGTACGTGGGG | 58% | 0 |
| (Gap Junction | (SEQ ID NO. 85) | | |
| Protein Alpha 8) | TACGGTTAGATCGTCTGACCTGG | 54% | 0 |
| (i.e., Cx 50) | (SEQ ID NO. 86) | | |
| | ACTAACGGCGGCCCGGACCAGGG | 54% | 3 |
| | (SEQ ID NO. 87) | | |
| PDE1 | GTCGATAAGCTGCACTCACGTGG | 63.9% | 0 |
| (Phosphodiesterase | (SEQ ID NO. 88) | | |
| type 1) | GCTGCATTATCGGTGGACCATGG | 63.15% | 1 |
| | (SEQ ID NO. 89) | | |
| | TGATCGGAAGTCAACCATGGTGG | 72.12% | 2 |
| | (SEQ ID NO. 90) | | |
| PDE2 | GTTCGACGAGGACCTGGCGACGG | 66.25% | 0 |
| (Phosphodiesterase | (SEQ ID NO. 91) | | |
| type 2) | GCGGATGGGCATATGCGTCAGGG | 64.96% | 1 |
| | (SEQ ID NO. 92) | | |
| | TCCGCTTTTCTACCGCGGCGTGG | 61.54% | 0 |
| | (SEQ ID NO. 93) | | |
| PDE3 | ACTGTGGACATCGCCGTCATGGG | 62.29% | 1 |
| (Phosphodiesterase | (SEQ ID NO. 94) | | |
| type 3) | TTCGTACATACCTGAATCACTGG | 52.32% | 0 |
| | (SEQ ID NO. 95) | | |
| | ATCGTTATATAGCACCGCCTAGG | 62.74% | 2 |
| | (SEQ ID NO. 96) | | |
| PDE4 | GGATGAGTTCTCGTCCCGGGAGG | 70.75% | 0 |
| (Phosphodiesterase | (SEQ ID NO. 97) | | |
| type 4) | CGCGGCTGCCATCCACGATGTGG | 59.51% | 1 |
| | (SEQ ID NO. 98) | | |
| | TTTTGCGTGTCGGATTACGCTGG | 54.85% | 2 |
| | (SEQ ID NO. 99) | | |
| PDE5 | TTGAACCGAGGATCCTAGTATGG | 43.66% | 1 |
| (Phosphodiesterase | (SEQ ID NO. 100) | | |
| type 5) | GCACGAGGACTCTGCTGCAAGGG | 59.37% | 3 |
| | (SEQ ID NO. 101) | | |
| | TACCCGTTGTTGAATAGGCCAGG | 47.69% | 1 |
| | (SEQ ID NO. 102) | | |
| PDE6 | GACCGCTTACTAAAGCCCAATGG | 68.41% | 2 |
| (Phosphodiesterase | (SEQ ID NO. 103) | | |
| type 6) | GACGATGTTCCAAAAGATCGTGG | 60.45% | 0 |
| | (SEQ ID NO. 104) | | |
| | CATCATGAATGCGCCTGCGGAGG | 57.78% | 0 |
| | (SEQ ID NO. 105) | | |
| PDE7 | TTGTAACCCATGTCGGACGTGGG | 67.77% | 1 |
| (Phosphodiesterase | (SEQ ID NO. 106) | | |
| type 7) | GTGAAGATCTAAGATATCGCTGG | 60.66% | 0 |
| | (SEQ ID NO. 107) | | |
| | TTTGTAACCCATGTCGGACGTGG | 52.56% | 0 |
| | (SEQ ID NO. 108) | | |
| PDE8 | TCCATGGCCCGAGCTATCCGTGG | 59.86% | 3 |
| (Phosphodiesterase | (SEQ ID NO. 109) | | |
| type 8) | CCGAGTCAACGCTAAGATCATGG | 52.62% | 0 |
| | (SEQ ID NO. 110) | | |
| | CGGTCTAGGGCTTCTGTCACAGG | 49.04% | 3 |
| | (SEQ ID NO. 111) | | |
| PDE9 | GGAGAACCACCACTGCGCCGTGG | 65% | 1 |
| (Phosphodiesterase | (SEQ ID NO. 112) | | |
| type 9) | TGAAGTCCCTGACCAGCCCGAGG | 70% | 0 |
| | (SEQ ID NO. 113) | | |
| | GGCTGTCCTAGAGAAACGCGTGG | 61% | 1 |
| | (SEQ ID NO. 114) | | |
| PDE10 | AGACCCCCAGATGGAAAGGAGGG | 83% | 1 |
| (Phosphodiesterase | (SEQ ID NO. 115) | | |
| type 10) | TTTGGAATCTGCCTGCTGAGCGG | 81% | 1 |
| | (SEQ ID NO. 116) | | |
| | GCAAGGCGCCGGGAGCACAGTGG | 42% | 0 |
| | (SEQ ID NO. 117) | | |
| PDE11 | GTAGCTACTGAATCTAGCATCGG | 93% | 2 |
| (Phosphodiesterase | (SEQ ID NO. 118) | | |
| type 11) | AATTTTTGATCGGAACCGGAAGG | 65% | 1 |
| | (SEQ ID NO. 119) | | |
| | GCAAAATGACTACAGCCTGAAGG | 46% | 0 |
| | (SEQ ID NO. 120) | | |
| miR-182-a | CTACCATTGCCAAAAACGGGGGG | 76% | 1 |
| | (SEQ ID NO. 121) | | |
| | GGTGAGGTAACAGGATCCGGTGG | 75% | 0 |
| | (SEQ ID NO. 122) | | |
| | TCTACCATTGCCAAAAACGGGGG | 45% | 2 |
| | (SEQ ID NO. 123) | | |
| PLCG1 | CCTATCCAGGTACCTGACCGGGG | 68.93% | 1 |
| Gamma 1) | (SEQ ID NO. 124) | | |
| (Phospholipase C | GAGCGTGTAGTCGCCCACGAAGG | 69.44% | 2 |
| | (SEQ ID NO. 125) | | |
| | GCGCTATCCCATCAACGAGGAGG | 67.28% | 0 |
| | (SEQ ID NO. 126) | | |

As shown in Table 1, the sgRNA sequences for CAPN3 may also result in knock-out of the CAPN3 isoforms Lp82 and Lp-83. See, e.g., L. Chen et al., “CAPN3: A muscle-specific calpain with an important role in the pathogenesis of diseases,” Intl. J. Mol. Med. Vol. 48, No. 5 (November 2021).

Table 2 below displays a list of possible knock-in and/or addition candidates mentioned in the present disclosure, along with specific sgRNA sequences for each candidate. PAM (protospacer adjacent motif) sequences are underlined.

TABLE 2
sgRNA sequences for knock-in and/or addition candidates.

| Knock-in candidates | sgRNA sequence (PAM underlined) |
|---|---|
| AAVS1 | GACAGAAAAGCCCCATCCTTAGG |
| (Adeno-associated virus | (SEQ ID NO. 127) |
| integration site 1) | ACTAGGAAGGAGGAGGCCTAAGG |
| | (SEQ ID NO. 128) |

Accordingly, embodiments of the present disclosure provide novel methods, devices, and systems that reduce the formation of proteins responsible for crowding, compacting, and/or causing increased internal lens pressure, thereby treating (e.g., retarding, eliminating, or reversing) one or more lens aggregation diseases (e.g., presbyopia, cataract).

Additional features of the aforementioned examples and embodiments of the present disclosure are presented in the following Examples, which should not be construed as limiting the present disclosure, which is defined by the claims.

### Example 1

This example relates to embodiments of the present disclosure that reduce the formation of proteins responsible for crowding, compacting, and/or causing increased internal lens pressure.

Generally, a human lymphatic endothelial cell (HLEC) line was grown in Dulbecco's Modified Eagle's Medium (DMEM) supplemented with 15% fetal bovine serum (FBS), 800 units per milliliter (U/mL/) penicillin, and 1 mg/mL streptomycin. Cells were cultured at 37° C. in 5% CO₂ and then split twice weekly using 0.25% trypsin. For the transfection experiments, passaged cells at 70-85% confluency were used.

Transfection was conducted with the Nucleofector™ I instrument and accompanying reagent kit (Amaxa, Cologne, Germany). Briefly, 1×10⁶ HLECs were suspended in 100 μL Cell Line Nucleofector™ Solution V at room temperature, followed by addition of 5 μg plasmid. The mixture of HLECs, Nucleofector™ solution, and plasmid was transferred to a 1 mm electroporation cuvette, which was then inserted into the Nucleofector™ I and reacted by means of the automated transfection program X-01. Immediately after transfection, 1000 μL of RMPI 1640 medium was added to the cuvette to reduce damage to the cells and the transfected cells were transferred to pre-warmed medium, supplemented with 15% FBS and plated onto six-well culture plates. The transfection efficiency was determined by observing the green fluorescent protein (GFP) expression under an inverted fluorescence microscope (Leica, Heerbrugg, Switzerland).

It should be appreciated that the aforementioned transfection may be achieved via, for example, the SF Cell Line 4D-Nucleofector™ X Kit S (Lonza, Switzerland). Lipofectamine transfection can also be used, with reagents and protocols using, e.g., the Lipofectamine 2000 Transfection Reagent (ThermoFisher Scientific, Massachusetts, United States). Another alternative includes, for instance, viral vectors (e.g., adeno-associated viruses (AAV)).

Culturing

To culture the cells, cells were grown in DMEM supplemented with 15% FBS, 800 U/mL penicillin, and 1 mg/mL streptomycin. Cells were cultured at 37° C. in 5% CO₂ and then split twice weekly using 0.25% trypsin.

Splitting Cells

Cells were split when they reached around 80-90% confluency (e.g., every two days). However, if cells are not sufficiently confluent, they may be split the next day. During the first cell split, cells may be counted first and split in order to be 2×10⁶ cells/mL.

To split adherent cells:

1. Carefully remove the media from the petri dish using a vacuum pump or a pipette.

2. Carefully add 7 mL of pre-warmed PBS to the wall (not directly on the cells) of the Petri dish to wash the cells.

3. Remove the PBS using a vacuum pump.

4. Add 1 mL of the trypsin directly to the cells.

5. Shake the plate to distribute the trypsin.

6. Incubate the plate at 37° C. for 5 min.

7. Add 9 mL of culture media (DMEM) to the plate. The FBS contained in the growing media will inactivate the trypsin.

8. Pipette up and down to disrupt the cell clumps. This results in achieving single cells which is important to seeding the cells and having an even distribution of cells in the well, and to ensure cell counting is accurate.

9. Place 9 mL of new DMEM in a new petri dish (e.g., 10 cm) and then take 1 mL of the detached cells from step 8. The cells can be split 1:10.

Cell Transfection Using Lipofectamine

Lipofectamine is a standard reagent for transfection in cell lines growing in adhesion. The transfection protocol is based on the recommendations of the Lipofectamine 2000® Reagent (Thermoscientific, Massachusetts, United States). It should be appreciated that there may be variations in the below when other protocols and/or reagents are used.

Block 1: Cell seeding (24 hours prior to transfection). To ensure a proper transfection (e.g., good efficiency and viability), cells should reach an appropriate confluency in the dish or well plate of choice of 70-90%.

10. Carefully remove the media from the petri dish using a vacuum pump or a pipette.

11. Carefully add 7 mL of pre-warmed PBS to the wall of the petri dish to wash the cells.

12. Remove the PBS using a vacuum pump.

13. Add 1 mL of trypsin directly to the cells.

14. Incubate the petri dish at 37° C. for 5 min.

15. Add 9 mL of growing media to the petri dish.

16. Pipette up and down to disrupt the cell clumps.

17. Count the cells using a counting chamber or any other method known in the art.

18. Assuming 1 million per mL of cell suspension, seed three wells containing 150,000 cells each (total of 450,000 cells) by taking 450 μL (or 0.45 mL) of cell suspension.

19. Pipette 450 μL into a 15 mL tube, fill the tube up to 5 mL with PBS, and then spin down the tube at 300 G for 5 minutes.

20. After centrifugation, there should be a pellet and a supernatant.

21. Remove the supernatant.

22. Resuspend the pellet in fresh warm media. The goal will be to fill three wells of a 24-well plate, each of which usually contains 500 μL of media. Therefore, the pellet can be resuspended in 1.5 mL of media to create a “cell master mix.”

23. Mix well with a pipette.

24. Add 500 μL of cell suspension to each well.

25. Swirl the plate (e.g., in a cross-like fashion) to ensure proper cell distribution.

26. Put the plate back in a 37° C. incubator for 24 hours.

27. After 24 hours, check cell density under a microscope.

28. In the case of lipofectamine, the transfection is divided into two preliminary steps. Generally, a cargo mix is first created, which contains the DNA, mRNA, and/or protein to be delivered to the cells. Required components are generally mixed in the same tube, and a master mix is prepared if possible. Second, a lipofectamine mix is created, in which the lipofectamine reagent is mixed with a free cell growth medium. Then the lipofectamine mix is mixed with the cargo mix.

29. To prepare the transfection reaction for a 24-well plate, first create mix #1. In a 1.5 ml tube mix together 900 ng of Cas9 encoding plasmid, 450 ng of sgRNA encoding plasmid and 300 ng of GFP encoding plasmid. Fill up the volume with free cell growth medium to 50 μL. Mix well with a pipette and spin down the tube with a quick pulse. This step could be performed under the cell hood to avoid contamination. It should be appreciated that it is not a requirement to add the plasmid encoding for GFP. This is in order to enable checking transfection efficiency by looking at the total number of GFP-positive cells. Further, other amounts of reagents can be used (e.g., 30 pmol of Cas9 and 100 pmol of sgRNA). Using CRISPR-Cas as mRNA or protein may increase efficiency.

30. Next create mix #2. In a separate tube, mix together 2 μL of Lipofectamine 2000® Reagent (Thermoscientific, Massachusetts, United States) with free cell growth medium. Mix well by pipetting and spinning down the tube. Let the tube rest at room temperature for 5 minutes.

31. Add 50 μL of mix #2 to mix #1 and pipette up and down gently. Then let the mixture rest for 20 minutes at room temperature. The final volume is 100 μL.

32. Add the entire 100 μL mixture drop-wise to the cells which have been seeded the day before. It should be appreciated that such drop-wise addition will help distribute the transfection reaction evenly.

33. Swirl the plate (e.g., in a cross-like fashion) to ensure all cells receive the transfection material.

34. Return the plate to the 37° C. incubator.

Block 2: Changing media (16 hours post-transfection). Note that transfection reagents may be toxic. Therefore, media should be replaced with fresh media after one day.

35. Carefully remove the media with a vacuum pump.

36. Carefully add fresh warm media to the wall of the well. Avoid dropping the media directly on the cells to avoid detaching them.

Block 3: Transfection check and cell split (48 hours to 72 hours post-transfection). To check for transfection efficiency, check the fraction of GFP-positive cells using, e.g., a standard flow cytometer. This assessment can be done 48 to 72 hours post-transfection to catch the peak of expression of GFP expressing plasmid. At this point, cells can also be checked to see if they have reached 80% to 90% confluency. If this is the case, cells should be detached, as done in step 1 above herein, and split 1:10 in a new well.

37. Detach the cells as done in step 1 above herein.

38. After resuspending the cells in new fresh media to inactivate the trypsin, take 50 μL of cell suspension and put in a well containing 450 μL of fresh medium. This results in splitting the cells in a 1:10 ratio.

39. Take 50 μL from the same cell suspension and put it in a 1.5 mL tube with 500 μL of PBS. Spin the tube down at 300 G for 5 minutes.

40. Remove the PBS and resuspend the pellet in 200 μL of FACS (Flow Cytometry Staining) Buffer (which is usually a mix of PBS and ethylenediaminetetraacetic acid (EDTA)). Spin down at 300 G for 5 minutes.

41. Use a flow cytometry device and measure the amount of GFP-positive cells. Depending on the GFP plasmid used and on the amount of time lapsed post-transfection, a 60% to 70% amount of GFP-positive cells indicates a good transfection efficiency. the Flow Cytometry device of choice and measure the amount of GFP+ cells.

Cell Transfection Using Nucleofection

This is an alternative method which may be used to increase the efficiency of editing if needed. By using an electric pulse, it is possible to open small pores in the cell membrane, which will facilitate the intake of the reagents within the cells. Specifically, nucleofection may permit a better delivery inside the nucleus. Electroporation can be applied to both adherent and suspension cells.

Block 1: Cell expansion. Although some protocols suggest “pre-seeding” cells, regardless of whether they are adherent or in suspension, before nucleofection, this is not necessarily a requirement. Generally, electroporation is performed on cells when they are in their growing phase. For this reason, it is important to grow the cells at the proper density. With HLEC, it is possible to try to grow them at, e.g., 2×10⁶/mL, which usually matches the timepoint at which the cells should be split.

Block 2: Counting the cells. Generally, these steps proceed similarly to those described above for lipofection (i.e., steps 10 through 17).

42. Cells can be counted using an appropriate method and/or system known in the art (e.g., a counting chamber, an automated system, or the like). For each reaction, 1000.000 for HLEC is required. For example, if counting indicates 2 million cells per mL, 1 mL of cell suspension is needed.

43. Take 1 mL of cell suspension and put in a tube (e.g., 1.5 mL tube). Place in a 37° C. incubator.

Block 3: Preparing the nucleofection reaction.

44. For this example, the protocol from the Lonza 4-D Nucleofector may be used. For mix #1, mix together the nucleofection solution (16.4 μL) and the supplemental solution (3.6 μL), for a final volume of 20 μL. The solution could be made under a chemical hood or other appropriate ventilation and then be placed at room temperature. It should be appreciated that other protocols (e.g., the SE kit program FF120 for HLEC) may be used.

45. To create mix #2, first mix together the DNA to be transfected in a tube. For instance, 900 ng of Cas9 encoding plasmid, 450 ng of sgRNA encoding plasmid, and 300 ng of GFP encoding plasmid may be mixed together. The DNA should be concentrated since the total DNA mix should not exceed 10% of the total reaction volume. The total DNA mix could preferably be 2 μL, although it is possible to add up to 5 μL. However, amounts above 5 μL may result in dilution of the nucleofection solution, which may hamper efficiency.

46. Prepare the strip containing the cuvettes for nucleofection.

47. Turn on the nucleofection device and select an appropriate program, which dictates the electric pulses (which are cell line-specific and provided by the kit provider).

48. Take the tube containing the 2 mL of cell suspension and add up to 1 mL of PBS.

49. Spin down the tube at 300 G for 5 minutes.

50. Remove the supernatant. Care should be taken since leftover media and/or PBS may dilute the nucleofection solution. Also, they may contain contaminants that can comprise efficiency of the nucleofection. Use a 20 μL pipette to remove the last few drops.

51. Use a 20 μL pipette to take up the nucleofection mix and resuspend the cells with it. Pipette gently to resuspend the cells.

52. Collect the cell suspension plus nucleofection mix and transfer it to the tube containing the DNA mix.

53. Mix well the cells, nucleofection mix, and DNA mix and transfer the mixture to the cuvette.

54. Place the cuvette into the electroporator and begin the program.

55. When the electroporation is complete, take a 48-well plate and add 100 μL of fresh warm media into an empty well. Then add 100 μL of fresh media to the cuvette where the cells were placed for nucleofection in order to recover them.

56. Pipette gently up and down and transfer to the well containing the 100 μL of fresh media

57. Place the 48-well plate back into the incubator.

Block 4: Media change. This is in order to remove residuals of the nucleofection solution, which can be toxic.

58. The following day, carefully remove 100 μL without touching the bottom of the well, to not disturb the cells.

59. Add 100 μL of fresh warm media and pipette gently up and down.

Block 5: Transfection check and cell split (48 hours to 72 hours post-transfection)

60. As in the case of lipofection, one may use a percentage of GFP-positive cells to indirectly verify efficiency of the nucleofection. If the 48-well plate is full, one may consider splitting the cells 1:2 in a 12-well plate.

61. Collect 20 μL of cell suspension in a separate tube and fill it up to 500 μL with PBS. Spin the tube down at 300 G for 5 minutes.

62. Resuspend the cells in 200 μL of FACS buffer.

63. Using a flow cytometry device, measure the amount of GFP-positive cells. Depending on the GFP plasmid used and the time post-transfection, a 60% to 70% yield of GFP-positive cells is a good transfection efficiency. A skilled artisan may notice higher toxicity compared to lipofection, since nucleofection is harsher on the cells.

The following methods may be used to analyze the editing at the DNA level. The first step may be to extract the genomic DNA from the previously transfected cells which will be then used as input for a polymerase chain reaction (PCR).

Block 1: Harvesting the cells for genomic extraction. To harvest the cells, one may use any commercially available kit available for such a purpose.

1. To harvest adherent cells like HLEC, they must be detached using trypsin, as stated above herein.

2. After harvesting the cells (e.g., adherent or suspension cells), spin the cells down at 300 G for 5 minutes.

3. Try to remove all the supernatant without touching the cells.

4. Place the tube containing the pellet on ice.

Block 2: Genomic DNA extraction. As previously stated above herein, there are different methods to extract genomic DNA. For example, there are kits like the QuickExtract kit (Lucigen), which allows for lysis of cells and genomic DNA extraction in less than 15 minutes. The genomic DNA obtained can then be immediately used for the following PCR steps. However, it should be appreciated that there will be residuals from buffer used to lysate the cells and extract the DNA. Accordingly, there are also filtered column-based methods known in the art, which allow for a cleaner genomic DNA, e.g., NucleoSpin Tissue Mini kit (Machery-Nagel), which takes longer (30 minutes) but guarantees a cleaner extract. Generally, any extraction method which guarantees an extract clean enough to be used as input for PCR should be acceptable.

Block 3: Mismatch-based assay (e.g., T7E1, Surveyor). The aim of the PCR is to amplify the CRISPR-targeted region, which then can be used for downstream assays. One such method is a mismatch-based assay like the T7E1 or the Surveyor assay. The obtained PCR product is then purified and denatured so that the resulting DNA is single-stranded. Then, the denatured DNA is re-annealed. During the reannealing steps, the strand containing mutations generated by CRISPR targeting (either insertion or deletion) may anneal with a strand which contains either a different insertion or deletion, or none at all. This will generate mismatches that create “bulges” where the mismatches are located. The T7E1 will cut where the bulge is located and, when the reaction is run on a gel, a restriction pattern will be observed if editing has occurred.

The first important step of the T7E1 assay is to obtain a clean and unspecific-free PCR product. This means that, ideally, at the end of the PCR, only one band should be observed on the gel. The second important step is that the product should not be bigger than 500-600 nucleotides and not shorter than 300 nucleotides. Therefore, specific primers should be designed. To this end, it is important to BLAST the primers before ordering them. A safe practice is also to design two-three different primer pairs and test all of them to see which one yields the best product. In order to increase the specificity of the PCR, one may also run a small-scale experiment (e.g., 12.5 μL PCR reaction) and test five-six different melting temperatures (Tm) if a thermocycler device with a gradient/step feature is used.

To set up the PCR reaction, one can use any standard manufacturer procedure for the polymerase used. Since the product to be amplified is in the range of 500-600 nucleotides maximum, a high-fidelity polymerase is not required. For the input of the PCR, a non-limiting example is to use 100 ng of extracted genomic DNA in a 50 μL PCR reaction.

When the PCR reaction is finished, load 10% (e.g., 5 μL) of the PCR reaction on a 1% agarose gel and check if only one band is observed. If only one band is seen, purification of the rest of the reaction (e.g., 45 μL) can proceed using, for instance, column-based PCR purification kits. Gel extraction is also possible, but the yield is usually lower and contaminants from the residual of the agarose gel may affect the following steps.

If more than one band is observed on the gel, two bands may indicate the presence of a large deletion. However, such an event is more likely to happen when one is cutting with two sgRNAs at the same time at the two sides of the targeted locus.

To perform the nuclease assay, the following steps may be used:

1. Mix 100 to 200 ng of PCR product with 2 μL of 10× NEB Buffer 2. Then, fill up to 19 μL with water.

2. Set a thermocycler instrument as follows.

First, anneal the PCR products using the following conditions.

TABLE 3
Conditions for annealing PCR products in a nuclease assay

| STEP | TEMPERATURE | | RAMP RATE | TIME |
|---|---|---|---|---|
| Initial Denaturation | 95° | C. | | 5 minutes |
| Annealing | 95-85° | C. | −2° C./second | |
| | 85-25° | C. | −0.1° C./second | |
| Hold | 4° | C. | | Hold |

Next, add the nuclease (e.g., T7 Endonuclease I) to the annealed PCR products as follows:

TABLE 4
Nuclease reaction conditions for annealed PCR products

| COMPONENT | 20 μl REACTION | |
|---|---|---|
| Annealed PCR product | 19 | μl |
| T7 Endonuclease I | 1 | μl |
| Incubation Time | 15 | minutes |
| Incubation Temperature | 37° | C. |

Next, after 15 minutes, one may run the product on a 2% agarose gel that includes a negative control (i.e., a control in which the nuclease was not added in any of the samples that underwent the denaturation/renaturation reactions).

What should be observed is that a restriction digestion pattern should be seen only in the CRISPR-treated sample. This emphasizes why it is important to begin with a clean PCR product, which makes it easier to discriminate between the unmodified band and the one corresponding to the nuclease activity. If multiple bands are already present in the untreated product, it would be harder to determine which one is the result of CRISPR and which one is simply an unspecific product.

To evaluate cleavage efficiency, one may use open source software libraries and/or applications such as ImageJ that calculates the intensity of each band. It is then possible to calculate the representation of the cleaved bands compared to the total in order to have a semi-quantitative calculation of the cleavage efficiency. It should be appreciated that the results obtained may be an underestimation, since a homozygous mutation (i.e., the same mutation on both strands) will not be cleaved by the nuclease (e.g., T7E1) since no mismatch occurs. Further, the result may be biased by the PCR primers used, since deletions spanning the primer binding site will not be amplified.

It should further be appreciated that an alternative to mismatched-based assays include software that use Sanger sequencing results to provide a picture of the editing outcome. This may also offer a more quantitative overview of the editing outcome. The workflow is similar to that set forth above, e.g., genomic DNA is first extracted and the target locus is amplified via PCR. At this point, no further enzymatic treatment is required, but the purified PCR product is sent for sequencing to the service provider of choice. The “.ab1” computer files obtained can be accessed and/or opened using open source software libraries and/or applications (e.g., TIDE or ICE). Generally, such software may use a few different pieces or instances of information to operate, specifically, the sgRNA sequence, the untreated sample “.ab1” file, and the treated sample “.ab1” file. At this point, the software may look for the sgRNA sequence in the untreated sample and establish the so called “editing window,” meaning the area within which any peaks (Sanger sequencing signals) diverge from the untreated sample. These peaks may be considered potential alterations introduced via CRISPR/Cas9. By comparing the untreated and the treated sample peaks, the software can discriminate insertions and deletions. Usually, such software can identify deletions spanning 50 nucleotides from the cut site and insertions up to 5 nucleotides.

Other software may be able to identify larger deletions and insertions; however, these results may be biased by the efficiency of the PCR at amplifying smaller fragments over larger ones. This means that the percentage of larger deletions could be overestimated. However, this may still be a valid tool to gain information about editing outcomes.

It should be noted that, regardless of the software libraries and/or applications used to analyze the “.ab1” file, there should ideally be a 200 nucleotide alignment between the untreated sample and the treated sample to reliably establish the editing window and properly determine real CRISPR related alterations. Accordingly, the cut site should be located at least 200 nucleotides downstream from the start of the amplicon sent for sequencing.

The following table contains possible primer sequences which can be used to amplify the target locus before running any of the analyses described above herein (e.g., the T7E1 analysis or the TIDE/ICE analysis). In the table, “Fw” refers to the forward direction and “Rv” refers to the reverse direction.

TABLE 5
Primer sequences for amplifying a target locus

| | | Melting | |
|---|---|---|---|
| | Sequence (5′ to 3′) and | temperature | Product site |
| Primer Target | identification number | (C.º) | (base pairs) |
| CRYAB sgRNA#1 | GATCACCATGAGCCTTAG | 60 | 532 |
| Fw | (SEQ ID NO. 1) | | |
| CRYAB sgRNA#1 | TCAAAGGCCACATCTCTG | | |
| Rv | (SEQ ID NO. 2) | | |
| CRYAB sgRNA#2 | TTCTCACTGGTAGGAGTG | 61 | 480 |
| Fw | (SEQ ID NO. 3) | | |
| CRYAB sgRNA#2 | AGCTTCAGCACTAGTCAC | | |
| Rv | (SEQ ID NO. 4) | | |
| CRYAB sgRNA#3 | TTCTCACTGGTAGGAGTG | 61 | 480 |
| Fw | (SEQ ID NO. 5) | | |
| CRYAB sgRNA#3 | AGCTTCAGCACTAGTCAC | | |
| Rv | (SEQ ID NO. 6) | | |
| CRYBA1 sgRNA#1 | GATTGTGCCACTGCACTC | 62 | 590 |
| Fw | (SEQ ID NO. 7) | | |
| CRYBA1 sgRNA#1 | CCATGGACATAACTGTTGC | | |
| Rv | (SEQ ID NO. 8) | | |
| CRYBA1 sgRNA#2 | GCTGGATTGGTTATGAGC | 61 | 393 |
| Fw | (SEQ ID NO. 9) | | |
| CRYBA1 sgRNA#2 | ACTGGGCTCTTGAGTATC | | |
| Rv | (SEQ ID NO. 10) | | |
| CRYBA1 sgRNA#3 | ACAAATAGTGCCTGACAC | 60 | 351 |
| Fw | (SEQ ID NO. 11) | | |
| CRYBA1 sgRNA#3 | GCTTAGCAAGGTCTCATG | | |
| Rv | (SEQ ID NO. 12) | | |
| CRYBA2 sgRNA#1 | CGGTCCATTTCCCTCGCG | 65 | 394 |
| Fw | (SEQ ID NO. 13) | | |
| CRYBA2 sgRNA#1 | GCCGTTTTCCACCTTGAC | | |
| Rv | (SEQ ID NO. 14) | | |
| CRYBA2 sgRNA#2 | CGGTCCATTTCCCTCGCG | 65 | 394 |
| Fw | (SEQ ID NO. 15) | | |
| CRYBA2 sgRNA#2 | GCCGTTTTCCACCTTGAC | | |
| Rv | (SEQ ID NO. 16) | | |
| CRYBA2 sgRNA#3 | CGGTCCATTTCCCTCGCG | 65 | 394 |
| Fw | (SEQ ID NO. 17) | | |
| CRYBA2 sgRNA#3 | GCCGTTTTCCACCTTGAC | | |
| Rv | (SEQ ID NO. 18) | | |
| CRYBA4 sgRNA#1 | TCCAGCCATCGTCAAGTG | 62 | 517 |
| Fw | (SEQ ID NO. 19) | | |
| CRYBA4 sgRNA#1 | AGAGTTTAAGGCAGGCTC | | |
| Rv | (SEQ ID NO. 20) | | |
| CRYBA4 sgRNA#2 | CACCTGTGCTGTCTAGTG | 59 | 430 |
| Fw | (SEQ ID NO. 21) | | |
| CRYBA4 sgRNA#2 | CACTCCTCAGTACTTCAG | | |
| Rv | (SEQ ID NO. 22) | | |
| CRYBA4 sgRNA#3 | TCCAGCCATCGTCAAGTG | 62 | 517 |
| Fw | (SEQ ID NO. 23) | | |
| CRYBA4 sgRNA#3 | AGAGTTTAAGGCAGGCTC | | |
| Rv | (SEQ ID NO. 24) | | |
| CRYBB1 sgRNA#1 | AACAGGCTCATCTCTCTC | 60 | 573 |
| Fw | (SEQ ID NO. 25) | | |
| CRYBB1 sgRNA#1 | CATCCAGCCTCATCTATC | | |
| Rv | (SEQ ID NO. 26) | | |
| CRYBB1 sgRNA#2 | TCACACAGCCTGTAAGTG | 59 | 507 |
| Fw | (SEQ ID NO. 27) | | |
| CRYBB1 sgRNA#2 | CTAGCTGGGTATGTAGTG | | |
| Rv | (SEQ ID NO. 28) | | |
| CRYBB1 sgRNA#3 | TCACACAGCCTGTAAGTG | 59 | 507 |
| Fw | (SEQ ID NO. 29) | | |
| CRYBB1 sgRNA#3 | CTAGCTGGGTATGTAGTG | | |
| Rv | (SEQ ID NO. 30) | | |
| CRYBB2 sgRNA#1 | GGCTTCACCCTTCCTAGTG | 66 | 348 |
| Fw | (SEQ ID NO. 31) | | |
| CRYBB2 sgRNA#2 | AGGTCTGGAGGGTTCCTG | | |
| Rv | (SEQ ID NO. 32) | | |
| CRYBB2 sgRNA#2 | GGCTTCACCCTTCCTAGTG | 66 | 348 |
| Fw | (SEQ ID NO. 33) | | |
| CRYBB2 sgRNA#3 | AGGTCTGGAGGGTTCCTG | | |
| Rv | (SEQ ID NO. 34) | | |
| CRYBB2 sgRNA#3 | GGCTTCACCCTTCCTAGTG | 66 | 348 |
| Fw | (SEQ ID NO. 35) | | |
| CRYBB2 sgRNA#3 | AGGTCTGGAGGGTTCCTG | | |
| Rv | (SEQ ID NO. 36) | | |
| CRYBB3 sgRNA#1 | AATGTAGGCAGGCAGAGTG | 61 | 386 |
| Fw | (SEQ ID NO. 37) | | |
| CRYBB3 sgRNA#2 | CCAGGCTTTATTGAGCAG | | |
| Rv | (SEQ ID NO. 38) | | |
| CRYBB3 sgRNA#2 | GCATCTGGAGCCTCCTTG | 60 | 436 |
| Fw | (SEQ ID NO. 39) | | |
| CRYBB3 sgRNA#3 | GACCTCTGTTCTGGATATG | | |
| Rv | (SEQ ID NO. 40) | | |
| CRYBB3 sgRNA#3 | GCATCTGGAGCCTCCTTG | 60 | 436 |
| Fw | (SEQ ID NO. 41) | | |
| CRYBB3 sgRNA#3 | GACCTCTGTTCTGGATATG | | |
| Rv | (SEQ ID NO. 42) | | |

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

I/We claim:

1. A method for reducing formation of proteins responsible for crowding, compacting, and/or causing increased lens pressure, the method comprising:
at least one of downregulating and deleting one or more genes that encode for one or more proteins responsible for at least one of crowding, compacting, and causing increased lens pressure,
wherein the one or more proteins comprise one or more crystallins selected from the group consisting of αB, βA4, βα3/α3, ββ2, ββ2P1, and combinations thereof.

2. The method of claim 1, wherein the at least one of downregulating and deleting is performed using one or more gene editing processes selected from the group consisting of clustered regularly interspaced short palindromic repeats (CRISPR), transcription activator-like effector nucleases (TALENs), retron library recombineering (RLR), Zinc finger nucleases (ZFNs), short synthetic single-stranded oligonucleotide modification, nucleofection, and combinations thereof.

3. The method of claim 1, wherein the one or more genes are selected from the group consisting of the epithelial αB lens crystallin promoter gene, the epithelial βA4 lens crystallin promoter gene, the epithelial βα3/α3 lens crystallin promoter gene, the epithelial ββ2 lens crystallin promoter gene, the epithelial ββ2P1 lens crystallin promoter gene, the p38 mitogen-activated protein kinase (MAP kinase) gene, the Crystallin-50 (Cx50) gene, and combinations thereof.

4. The method of claim 1, further comprising administering gene therapy to a patient with one or more lens conditions, wherein the gene therapy utilizes at least one of donor deoxyribonucleic acid (DNA) and a CRISPR/Cas9 complex.

5. The method of claim 1, further comprising administering one or more compounds to a patient with one or more lens conditions, wherein the one or more compounds comprises an inhibitor that at least one of reduces and inhibits expression of the one or more genes.

6. The method of claim 5, wherein the inhibitor is configured to knock down activating at least one of transcription factor 4 (ATF4) and C/EBP homologous protein (CHOP).

7. The method of claim 5, wherein the one or more compounds comprises one or more protein chaperones selected from the group consisting of an alpha-crystallin protein, a beta-crystallin protein, a gamma crystallin protein, one or more proteins from within the heat shock proteins (Hsps), and combinations thereof.

8. A method for at least one of maintaining and reducing internal lens hydrostatic pressure by at least one of increasing gap junction coupling and reducing calcium from accumulating within a lens, the method comprising:
at least one of upregulating and adding one or more proteins that protect or enhance function of at least one of mammalian fibroblast growth factor (FGF) and one or more genes.

9. The method of claim 8, wherein the one or more proteins are selected from the group consisting of extracellular signal-regulated kinase (ERK), aquaporin-0, one or more PKC isotypes, calpastatin, phenylmethylsulfonyl fluoride (PMSF), leupeptin, α3 connexin, and combinations thereof.

10. The method of claim 8, wherein the one or more genes encode for a protein selected from the group consisting of: protein kinase A (PKA), cyclic AMP (cAMP), plasma membrane calcium ATPase (PMCA) 1, PMCA2, PMCA3, PMCA4, FGF receptor (FGFR) 1, FGFR2, FGFR3, FGFR4, aquaporin-1, aquaporin-5, phenylmethylsulfonyl fluoride (PMSF), inhibitor compound BIRB796, one or more forms of filensin, phakinin, one or more inwardly rectifying potassium channel (IRPC) proteins, pigment epithelium-derived factor (PEDF), CaM-dependent phosphodiesterase, adenylate cyclase, one or more phosphodiesterases, calsequestrin, triadin, junctin, and combinations thereof.

11. The method of claim 8, wherein the one or more genes upregulate expression levels of sarcoplasmic endoplasmic reticulum calcium ATPase (SERCA).

12. The method of claim 8, further comprising at least one of downregulating and deleting one or more genes selected from the group consisting of cyclic AMP (cAMP) responsive element modulator (CREM), protein kinase C (PKC) γ, a gene expressing MicroRNA-182-5p, a gene expressing calmodulin, a gene expressing nitric oxide synthetase, a gene expressing inositol 1,4,5-trisphosphate 3-kinases (IP3K), a gene expressing phospholipase C (PLC), a gene expressing cAMP-dependent phosphodiesterase, a gene expressing G-protein, a gene expressing decorin (DCN), and combinations thereof.

13. The method of claim 8, further comprising at least one of downregulating and deleting a gene expressing Cx50, wherein the one or more genes comprises a gene expressing Cx46.

14. The method of claim 8, further comprising using CGP54345 to inhibit PKC α.

15. The method of claim 8, further comprising increasing cyclic AMP (cAMP) levels using at least one of one or more chemicals and molecules, wherein the at least one of one or more chemicals and molecules comprise one or more phosphodiesterase (PDE) inhibitors.

16. A method for reducing internal lens hydrostatic pressure, the method comprising:
decreasing phosphorylation of one or more crystallins by altering one or more portions of the p38 mitogen-activated protein kinase (MAP kinase) pathway, wherein the altering is achieved by at least one of downregulating and deleting one or more genes in the p38 MAP kinase pathway.

17. The method of claim 16, further comprising at least one of reducing and deleting production of the one or more crystallins.

18. The method of claim 16, further comprising delivering one or more p38 MAP kinase inhibitors to a lens.

19. The method of claim 16, further comprising decreasing phosphorylation of one or more connexins.

20. A method for reducing formation of cataracts, the method comprising:
at least one of downregulating and deleting at least one of one or more genes and one or more proteases that destroy one or more gap junction proteins,
wherein the one or more genes are selected from the group consisting of a gene encoding for a calpain, a gene encoding for a calpain isoform, a gene encoding for transglutaminase (TGase), a gene encoding for a calcium-dependent protease, a gene encoding for an m-calpain, a gene encoding for an enzyme protease, a gene encoding for a Src kinase, a gene encoding for a myosin light-chain (MLC) kinase, and combinations thereof.

21. The method of claim 20, further comprising at least one of upregulating and adding a gene encoding for calpastatin.

22. The method of claim 20, further comprising adding one or more calpain inhibitors.


SEQUENCE LISTING (128 sequences)

SEQ ID NO: 1 - DNA, 18 bp, Artificial Sequence
  CRYAB sgRNA-1 forward primer
gatcaccatgagccttag

SEQ ID NO: 2 - DNA, 18 bp, Artificial Sequence
  CRYAB sgRNA-1 reverse primer
tcaaaggccacatctctg

SEQ ID NO: 3 - DNA, 18 bp, Artificial Sequence
  CRYAB sgRNA-2 forward primer
ttctcactggtaggagtg

SEQ ID NO: 4 - DNA, 18 bp, Artificial Sequence
  CRYAB sgRNA-2 reverse primer
agcttcagcactagtcac

SEQ ID NO: 5 - DNA, 18 bp, Artificial Sequence
  CRYAB sgRNA-3 forward primer
ttctcactggtaggagtg

SEQ ID NO: 6 - DNA, 18 bp, Artificial Sequence
  CRYAB sgRNA-3 reverse primer
agcttcagcactagtcac

SEQ ID NO: 7 - DNA, 18 bp, Artificial Sequence
  CRYBA1 sgRNA-1 forward primer
gattgtgccactgcactc

SEQ ID NO: 8 - DNA, 19 bp, Artificial Sequence
  CRYBA1 sgRNA-1 reverse primer
ccatggacataactgttgc

SEQ ID NO: 9 - DNA, 18 bp, Artificial Sequence
  CRYBA1 sgRNA-2 forward primer
gctggattggttatgagc

SEQ ID NO: 10 - DNA, 18 bp, Artificial Sequence
  CRYBA1 sgRNA-2 reverse primer
actgggctcttgagtatc

SEQ ID NO: 11 - DNA, 18 bp, Artificial Sequence
  CRYBA1 sgRNA-3 forward primer
acaaatagtgcctgacac

SEQ ID NO: 12 - DNA, 18 bp, Artificial Sequence
  CRYBA1 sgRNA-3 reverse primer
gcttagcaaggtctcatg

SEQ ID NO: 13 - DNA, 18 bp, Artificial Sequence
  CRYBA2 sgRNA-1 forward primer
cggtccatttccctcgcg

SEQ ID NO: 14 - DNA, 18 bp, Artificial Sequence
  CRYBA2 sgRNA-1 reverse primer
gccgttttccaccttgac

SEQ ID NO: 15 - DNA, 18 bp, Artificial Sequence
  CRYBA2 sgRNA-2 forward primer
cggtccatttccctcgcg

SEQ ID NO: 16 - DNA, 18 bp, Artificial Sequence
  CRYBA2 sgRNA-2 reverse primer
gccgttttccaccttgac

SEQ ID NO: 17 - DNA, 18 bp, Artificial Sequence
  CRYBA2 sgRNA-3 forward primer
cggtccatttccctcgcg

SEQ ID NO: 18 - protein, 18 aa, Artificial Sequence
  CRYBA2 sgRNA-3 reverse primer
GCCGTTTTCCACCTTGAC

SEQ ID NO: 19 - protein, 18 aa, Artificial Sequence
  CRYBA4 sgRNA-1 forward primer
TCCAGCCATCGTCAAGTG

SEQ ID NO: 20 - DNA, 18 bp, Artificial Sequence
  CRYBA4 sgRNA-1 reverse primer
agagtttaaggcaggctc

SEQ ID NO: 21 - DNA, 18 bp, Artificial Sequence
  CRYBA4 sgRNA-2 forward primer
cacctgtgctgtctagtg

SEQ ID NO: 22 - DNA, 18 bp, Artificial Sequence
  CRYBA4 sgRNA-2 reverse primer
cactcctcagtacttcag

SEQ ID NO: 23 - DNA, 18 bp, Artificial Sequence
  CRYBA4 sgRNA-3 forward primer
tccagccatcgtcaagtg

SEQ ID NO: 24 - DNA, 18 bp, Artificial Sequence
  CRYBA4 sgRNA-3 reverse primer
agagtttaaggcaggctc

SEQ ID NO: 25 - DNA, 18 bp, Artificial Sequence
  CRYBB1 sgRNA-1 forward primer
aacaggctcatctctctc

SEQ ID NO: 26 - DNA, 18 bp, Artificial Sequence
  CRYBB1 sgRNA-1 reverse primer
catccagcctcatctatc

SEQ ID NO: 27 - DNA, 18 bp, Artificial Sequence
  CRYBB1 sgRNA-2 forward primer
tcacacagcctgtaagtg

SEQ ID NO: 28 - DNA, 18 bp, Artificial Sequence
  CRYBB1 sgRNA-2 forward primer
ctagctgggtatgtagtg

SEQ ID NO: 29 - DNA, 18 bp, Artificial Sequence
  CRYBB1 sgRNA-3 forward primer
tcacacagcctgtaagtg

SEQ ID NO: 30 - DNA, 18 bp, Artificial Sequence
  CRYBB1 sgRNA-3 reverse primer
ctagctgggtatgtagtg

SEQ ID NO: 31 - DNA, 19 bp, Artificial Sequence
  CRYBB2 sgRNA-1 forward primer
ggcttcacccttcctagtg

SEQ ID NO: 32 - DNA, 18 bp, Artificial Sequence
  CRYBB2 sgRNA-2 reverse primer
aggtctggagggttcctg

SEQ ID NO: 33 - DNA, 19 bp, Artificial Sequence
  CRYBB2 sgRNA-2 forward primer
ggcttcacccttcctagtg

SEQ ID NO: 34 - DNA, 18 bp, Artificial Sequence
  CRYBB2 sgRNA-3 reverse primer
aggtctggagggttcctg

SEQ ID NO: 35 - DNA, 19 bp, Artificial Sequence
  CRYBB2 sgRNA-3 forward primer
ggcttcacccttcctagtg

SEQ ID NO: 36 - DNA, 18 bp, Artificial Sequence
  CRYBB2 sgRNA-3 reverse primer
aggtctggagggttcctg

SEQ ID NO: 37 - DNA, 19 bp, Artificial Sequence
  CRYBB3 sgRNA-1 forward primer
aatgtaggcaggcagagtg

SEQ ID NO: 38 - DNA, 18 bp, Artificial Sequence
  CRYBB3 sgRNA-2 reverse primer
ccaggctttattgagcag

SEQ ID NO: 39 - DNA, 18 bp, Artificial Sequence
  CRYBB3 sgRNA-2 forward primer
gcatctggagcctccttg

SEQ ID NO: 40 - DNA, 19 bp, Artificial Sequence
  CRYBB3 sgRNA-3 reverse primer
gacctctgttctggatatg

SEQ ID NO: 41 - DNA, 18 bp, Artificial Sequence
  CRYBB3 sgRNA-3 forward primer
gcatctggagcctccttg

SEQ ID NO: 42 - DNA, 19 bp, Artificial Sequence
  CRYBB3 sgRNA-3 reverse primer
gacctctgttctggatatg

SEQ ID NO: 43 - DNA, 23 bp, Artificial Sequence
  CALM1 (Calmodulin)
cgtcacgtcatgacaaacttagg

SEQ ID NO: 44 - DNA, 23 bp, Artificial Sequence
  CALM1 (Calmodulin)
gacggacaagtcaactatgaagg

SEQ ID NO: 45 - DNA, 23 bp, Artificial Sequence
  CALM1 (Calmodulin)
gacggacaagtcaactatgaagg

SEQ ID NO: 46 - DNA, 23 bp, Artificial Sequence
  NOS1 (Nitric Oxide Synthetase)
cgtattcatcagccggatgaggg

SEQ ID NO: 47 - DNA, 23 bp, Artificial Sequence
  NOS1(Nitric Oxide Synthetase)
ggtcacttcgtacgttcgcaggg

SEQ ID NO: 48 - DNA, 23 bp, Artificial Sequence
  NOS1(Nitric Oxide Synthetase)
tgtccgaaggcgcaaaagtgagg

SEQ ID NO: 49 - DNA, 23 bp, Artificial Sequence
  MLCK (Myosin Light Chain Kinase)
tcgcgatttagaagttgtggagg

SEQ ID NO: 50 - DNA, 23 bp, Artificial Sequence
  MLCK (Myosin Light Chain Kinase)
cgcgatttagaagttgtggaggg

SEQ ID NO: 51 - DNA, 23 bp, Artificial Sequence
  MLCK (Myosin Light Chain Kinase)
cgtcctcatcgtagtctatctgg

SEQ ID NO: 52 - DNA, 23 bp, Artificial Sequence
  PI3KCA (Phosphatidylinositol 3-kinase)
ggatttagctattcccacgcagg

SEQ ID NO: 53 - DNA, 23 bp, Artificial Sequence
  PI3KCA (Phosphatidylinositol 3-kinase)
tagctattcccacgcaggactgg

SEQ ID NO: 54 - DNA, 23 bp, Artificial Sequence
  PI3KCA (Phosphatidylinositol 3-kinase)
tcgaatatttacattcacgtagg

SEQ ID NO: 55 - DNA, 23 bp, Artificial Sequence
  CAPN1 (Calpain 1)
ggcaaataggtgaactaccgagg

SEQ ID NO: 56 - DNA, 23 bp, Artificial Sequence
  CAPN1 (Calpain 1)
aatggccatccgcatctcgtagg

SEQ ID NO: 57 - DNA, 23 bp, Artificial Sequence
  CAPN1 (Calpain 1)
cgcggtctacgaggtcaggaggg

SEQ ID NO: 58 - DNA, 23 bp, Artificial Sequence
  CAPN2 (Calpain 2)
agcggttgagcacctcccggagg

SEQ ID NO: 59 - DNA, 23 bp, Artificial Sequence
  CAPN2 (Calpain 2)
gtactcggtcaccggagccgagg

SEQ ID NO: 60 - DNA, 23 bp, Artificial Sequence
  CAPN2 (Calpain 2)
accgagcaacgatgacttggtgg

SEQ ID NO: 61 - DNA, 23 bp, Artificial Sequence
  CAPN3  (includes isoforms Lp82 and Lp85)
gagcgcaatcatgctacggcagg

SEQ ID NO: 62 - DNA, 23 bp, Artificial Sequence
  CAPN3  (includes isoforms Lp82 and Lp85)
tcgcatctcgtagctgttgatgg

SEQ ID NO: 63 - DNA, 23 bp, Artificial Sequence
  CAPN3  (includes isoforms Lp82 and Lp85)
ccgtagcatgattgcgctcatgg

SEQ ID NO: 64 - DNA, 23 bp, Artificial Sequence
  CAPN10 (Calpain)
gagtttgacgagctcaccgttgg

SEQ ID NO: 65 - DNA, 23 bp, Artificial Sequence
  CAPN10 (Calpain)
caccgttggctacccggtcacgg

SEQ ID NO: 66 - DNA, 23 bp, Artificial Sequence
  CAPN10 (Calpain)
ctacgccaagtgcgtgtgctggg

SEQ ID NO: 67 - DNA, 23 bp, Artificial Sequence
  TGM2 (Transglutaminase (TGase) 2)
gttgtagttggtcacgacgcggg

SEQ ID NO: 68 - DNA, 23 bp, Artificial Sequence
  TGM2 (Transglutaminase (TGase) 2)
tgcggaggtcaatgccgacgtgg

SEQ ID NO: 69 - DNA, 23 bp, Artificial Sequence
  TGM2 (Transglutaminase (TGase) 2)
acgctgccgatccaggacatggg

SEQ ID NO: 70 - DNA, 23 bp, Artificial Sequence
  PRKCG (Protein Kinase C Gamma)
cacgaagtcggggttcacgtagg

SEQ ID NO: 71 - DNA, 23 bp, Artificial Sequence
  PRKCG (Protein Kinase C Gamma)
gaaccccgacttcgtgcacccgg

SEQ ID NO: 72 - DNA, 23 bp, Artificial Sequence
  PRKCG (Protein Kinase C Gamma)
tgacaagttcttcacgcgggcgg

SEQ ID NO: 73 - DNA, 23 bp, Artificial Sequence
  cAMP
gcggtagaggttagcatccgagg

SEQ ID NO: 74 - DNA, 23 bp, Artificial Sequence
  cAMP
ctatagatggcatcaaccagcgg

SEQ ID NO: 75 - DNA, 23 bp, Artificial Sequence
  cAMP
cagctggtgaagcggtgtatggg

SEQ ID NO: 76 - DNA, 23 bp, Artificial Sequence
  p38
cacaaaaacggggttacgtgtgg

SEQ ID NO: 77 - DNA, 23 bp, Artificial Sequence
  p38
agctcctgccggtagaacgtggg

SEQ ID NO: 78 - DNA, 23 bp, Artificial Sequence
  p38
ataatggccgagctgttgactgg

SEQ ID NO: 79 - DNA, 23 bp, Artificial Sequence
  Src Kinase
gccgagcccaagctgttcggagg

SEQ ID NO: 80 - DNA, 23 bp, Artificial Sequence
  Src Kinase
cgatctgcaggcggaaccgtggg

SEQ ID NO: 81 - DNA, 23 bp, Artificial Sequence
  Src Kinase
ctatgactatgagtctaggacgg

SEQ ID NO: 82 - DNA, 23 bp, Artificial Sequence
  MLCK/MYLK
tcgcgatttagaagttgtggagg

SEQ ID NO: 83 - DNA, 23 bp, Artificial Sequence
  MLCK/MYLK
cgcgatttagaagttgtggaggg

SEQ ID NO: 84 - DNA, 23 bp, Artificial Sequence
  MLCK/MYLK
cgtcctcatcgtagtctatctgg

SEQ ID NO: 85 - DNA, 23 bp, Artificial Sequence
  GJA8 (Gap Junction Protein Alpha 8)
ccccgtccctgatgtacgtgggg

SEQ ID NO: 86 - DNA, 23 bp, Artificial Sequence
  GJA8 (Gap Junction Protein Alpha 8)
tacggttagatcgtctgacctgg

SEQ ID NO: 87 - DNA, 23 bp, Artificial Sequence
  GJA8 (Gap Junction Protein Alpha 8)
actaacggcggcccggaccaggg

SEQ ID NO: 88 - DNA, 23 bp, Artificial Sequence
  PDE1 (Phosphodiesterase type 1)
gtcgataagctgcactcacgtgg

SEQ ID NO: 89 - DNA, 23 bp, Artificial Sequence
  PDE1 (Phosphodiesterase type 1)
gctgcattatcggtggaccatgg

SEQ ID NO: 90 - DNA, 23 bp, Artificial Sequence
  PDE1 (Phosphodiesterase type 1)
tgatcggaagtcaaccatggtgg

SEQ ID NO: 91 - DNA, 23 bp, Artificial Sequence
  PDE2 (Phosphodiesterase type 2)
gttcgacgaggacctggcgacgg

SEQ ID NO: 92 - DNA, 22 bp, Artificial Sequence
  PDE2 (Phosphodiesterase type 2)
cggatgggcatatgcgtcaggg

SEQ ID NO: 93 - DNA, 23 bp, Artificial Sequence
  PDE2 (Phosphodiesterase type 2)
tccgcttttctaccgcggcgtgg

SEQ ID NO: 94 - DNA, 23 bp, Artificial Sequence
  PDE3 (Phosphodiesterase type 3)
actgtggacatcgccgtcatggg

SEQ ID NO: 95 - DNA, 23 bp, Artificial Sequence
  PDE3 (Phosphodiesterase type 3)
ttcgtacatacctgaatcactgg

SEQ ID NO: 96 - DNA, 23 bp, Artificial Sequence
  PDE3 (Phosphodiesterase type 3)
atcgttatatagcaccgcctagg

SEQ ID NO: 97 - DNA, 23 bp, Artificial Sequence
  PDE4 (Phosphodiesterase type 4)
ggatgagttctcgtcccgggagg

SEQ ID NO: 98 - DNA, 23 bp, Artificial Sequence
  PDE4 (Phosphodiesterase type 4)
cgcggctgccatccacgatgtgg

SEQ ID NO: 99 - DNA, 23 bp, Artificial Sequence
  PDE4 (Phosphodiesterase type 4)
ttttgcgtgtcggattacgctgg

SEQ ID NO: 100 - DNA, 23 bp, Artificial Sequence
  PDE5 (Phosphodiesterase type 5)
ttgaaccgaggatcctagtatgg

SEQ ID NO: 101 - DNA, 23 bp, Artificial Sequence
  PDE5 (Phosphodiesterase type 5)
gcacgaggactctgctgcaaggg

SEQ ID NO: 102 - DNA, 23 bp, Artificial Sequence
  PDE5 (Phosphodiesterase type 5)
tacccgttgttgaataggccagg

SEQ ID NO: 103 - DNA, 23 bp, Artificial Sequence
  PDE6 (Phosphodiesterase type 6)
gaccgcttactaaagcccaatgg

SEQ ID NO: 104 - DNA, 23 bp, Artificial Sequence
  PDE6 (Phosphodiesterase type 6)
gacgatgttccaaaagatcgtgg

SEQ ID NO: 105 - DNA, 23 bp, Artificial Sequence
  PDE6 (Phosphodiesterase type 6)
catcatgaatgcgcctgcggagg

SEQ ID NO: 106 - DNA, 23 bp, Artificial Sequence
  PDE7 (Phosphodiesterase type 7)
ttgtaacccatgtcggacgtggg

SEQ ID NO: 107 - DNA, 23 bp, Artificial Sequence
  PDE7 (Phosphodiesterase type 7)
gtgaagatctaagatatcgctgg

SEQ ID NO: 108 - DNA, 23 bp, Artificial Sequence
  PDE7 (Phosphodiesterase type 7)
tttgtaacccatgtcggacgtgg

SEQ ID NO: 109 - DNA, 23 bp, Artificial Sequence
  PDE8 (Phosphodiesterase type 8)
tccatggcccgagctatccgtgg

SEQ ID NO: 110 - DNA, 23 bp, Artificial Sequence
  PDE8 (Phosphodiesterase type 8)
ccgagtcaacgctaagatcatgg

SEQ ID NO: 111 - DNA, 23 bp, Artificial Sequence
  PDE8 (Phosphodiesterase type 8)
cggtctagggcttctgtcacagg

SEQ ID NO: 112 - DNA, 23 bp, Artificial Sequence
  PDE9 (Phosphodiesterase type 9)
ggagaaccaccactgcgccgtgg

SEQ ID NO: 113 - DNA, 23 bp, Artificial Sequence
  PDE9 (Phosphodiesterase type 9)
tgaagtccctgaccagcccgagg

SEQ ID NO: 114 - DNA, 23 bp, Artificial Sequence
  PDE9 (Phosphodiesterase type 9)
ggctgtcctagagaaacgcgtgg

SEQ ID NO: 115 - DNA, 23 bp, Artificial Sequence
  PDE10 (Phosphodiesterase type 10)
agacccccagatggaaaggaggg

SEQ ID NO: 116 - DNA, 23 bp, Artificial Sequence
  PDE10 (Phosphodiesterase type 10)
tttggaatctgcctgctgagcgg

SEQ ID NO: 117 - DNA, 23 bp, Artificial Sequence
  PDE10 (Phosphodiesterase type 10)
gcaaggcgccgggagcacagtgg

SEQ ID NO: 118 - DNA, 23 bp, Artificial Sequence
  PDE11 (Phosphodiesterase type 11)
gtagctactgaatctagcatcgg

SEQ ID NO: 119 - DNA, 23 bp, Artificial Sequence
  PDE11 (Phosphodiesterase type 11)
aatttttgatcggaaccggaagg

SEQ ID NO: 120 - DNA, 23 bp, Artificial Sequence
  PDE11 (Phosphodiesterase type 11)
gcaaaatgactacagcctgaagg

SEQ ID NO: 121 - DNA, 23 bp, Artificial Sequence
  miR-182-a
ctaccattgccaaaaacgggggg

SEQ ID NO: 122 - DNA, 23 bp, Artificial Sequence
  miR-182-a
ggtgaggtaacaggatccggtgg

SEQ ID NO: 123 - DNA, 23 bp, Artificial Sequence
  miR-182-a
tctaccattgccaaaaacggggg

SEQ ID NO: 124 - DNA, 23 bp, Artificial Sequence
  PLCG1 (Phospholipase C Gamma 1)
cctatccaggtacctgaccgggg

SEQ ID NO: 125 - DNA, 23 bp, Artificial Sequence
  PLCG1 (Phospholipase C Gamma 1)
gagcgtgtagtcgcccacgaagg

SEQ ID NO: 126 - DNA, 23 bp, Artificial Sequence
  PLCG1 (Phospholipase C Gamma 1)
gcgctatcccatcaacgaggagg

SEQ ID NO: 127 - DNA, 23 bp, Artificial Sequence
  AAVS1 (Adeno-associated virus integration site 1)
gacagaaaagccccatccttagg

SEQ ID NO: 128 - DNA, 23 bp, Artificial Sequence
  AAVS1 (Adeno-associated virus integration site 1)
actaggaaggaggaggcctaagg